(19) United States
(12) Patent Application Publication
(10) Pub. No.: US 2025/0322965 A1
(43) Pub. Date: Oct. 16, 2025

(54) METHODS AND SYSTEMS FOR PLANNING, PREDICTING, AND MONITORING THERAPIES FOR PULMONARY DISEASES

(71) Applicant: Apreo Health, Inc., Menlo Park, CA (US)

(72) Inventors: Martin L. Mayse, Wayzata, MN (US); Jagannath Padmanabhan, San Carlos, CA (US); Claudia Lynn Pham, III, San Francisco, CA (US); Patrick P. Wu, San Carlos, CA (US); Surag Mantri, East Palo Alto, CA (US); Allison Sihan Jia, San Francisco, CA (US); Karun D. Naga, Los Altos, CA (US)

(21) Appl. No.: 19/251,422

(22) Filed: Jun. 26, 2025

Related U.S. Application Data
(63) Continuation of application No. PCT/US2023/086498, filed on Dec. 29, 2023.
(60) Provisional application No. 63/477,623, filed on Dec. 29, 2022.

(51) Int. Cl. G16H 50/50; A61B 6/50; A61F 2/04

(52) U.S. Cl. CPC G16H 50/50; A61F 2/04; A61B 6/50; A61F 2002/043

(57) ABSTRACT

Methods for planning, predictive modeling, and monitoring therapies for pulmonary diseases are provided. In some embodiments, a method for planning a treatment for a patient having a pulmonary disease includes receiving patient data including computed tomography (CT) data of a lung of the patient. The method can include generating a set of lung metrics by inputting the patient data into a first machine learning algorithm. The method can also include predicting a response of the patient to treatment for the pulmonary disease by inputting the set of lung metrics into a second machine learning algorithm. The method can further include evaluating whether the patient is a candidate for the treatment for the pulmonary disease, based on the predicted response. The method can further include evaluating whether the patient has benefited following treatment and whether additional treatment is warranted.

### CROSS-REFERENCE TO RELATED APPLICATION

This application is a continuation of International Application No. PCT/US2023/086498, filed Dec. 29, 2023, which claims the benefit of priority to U.S. Patent Application No. 63/477,623, filed Dec. 29, 2023. Each of the foregoing applications is incorporated herein by reference in its entirety.

### TECHNICAL FIELD

The present technology generally relates to treatment planning, and in particular, to methods and systems for planning, predicting, and monitoring therapies for pulmonary diseases.

### BACKGROUND

Chronic obstructive pulmonary disorder (COPD) is a disease of impaired lung function. Symptoms of COPD include coughing, wheezing, shortness of breath, and chest tightness. Cigarette smoking is the leading cause of COPD, but long-term exposure to other lung irritants (e.g., air pollution, chemical fumes, dust) may also cause or contribute to COPD. In most cases, COPD is a progressive disease that worsens over the course of many years. Accordingly, many people have COPD, but are unaware of its progression. COPD is currently a major cause of death and disability in the United States. Severe COPD may prevent a patient from performing even basic activities such as walking, climbing stairs, or bathing. Unfortunately, there is no known cure for COPD. Nor are there known medical techniques capable of reversing the pulmonary damage associated with COPD. Conventional approaches to treating COPD are associated with serious complications, have limited effectiveness, are only suitable for a small percentage of COPD patients, and/or have other significant disadvantages. Given the prevalence of the disease and the inadequacy of conventional treatments, there is a great need for innovation in this field.

### SUMMARY

The subject technology is illustrated, for example, according to various aspects described below, including with reference to FIGS. 1-28. Various examples of aspects of the subject technology are described as numbered examples (1, 2, 3, etc.) for convenience. These are provided as examples and do not limit the subject technology.

Example 1. A method for planning a treatment for a patient having a pulmonary disease, the method comprising:
receiving patient data including computed tomography (CT) data of a lung of the patient;
generating a set of lung metrics by inputting the patient data into a first machine learning algorithm, wherein the set of lung metrics represents a state of the lung of the patient;
predicting a response of the patient to a treatment for the pulmonary disease by inputting the set of lung metrics into a second machine learning algorithm; and
evaluating whether the patient is a candidate for the treatment for the pulmonary disease, based on the predicted response.

Example 2. The method of example 1, wherein the patient data comprises one or more of the following: questionnaire information, medical record information, magnetic resonance imaging (MRI) data, single-photon emission computed tomography (SPECT) data, bronchoscopy data, ventilation-perfusion data, pulmonary function test data, chest radiography data, fluoroscopy data, photographs, or sensor data.

Example 3. The method of example 1 or 2, wherein the CT data comprises expiratory CT data.

Example 4. The method of example 3, wherein the CT data comprises inspiratory CT data.

Example 5. The method of any one of examples 1-4, wherein the patient data comprises data obtained at a plurality of different time points.

Example 6. The method of any one of examples 1-5, wherein the set of lung metrics correlates to whether the patient has at least one of chronic obstructive pulmonary disease (COPD), severe emphysema, or severe emphysema with hyperinflation.

Example 7. The method of any one of examples 1-6, wherein the set of lung metrics characterizes one or more lung parameters, the one or more lung parameters comprising one or more of the following: forced expiratory volume in 1 second, forced vital capacity, vital capacity, inspiratory capacity, inspiratory capacity/total lung capacity ratio, functional residual capacity, total lung capacity, diffusion capacity for carbon monoxide, residual volume, residual volume/total lung capacity ratio, closing volume, lobar and/or segmental tissue destruction, lobar and/or segmental air trapping, lobar and/or segmental fissure status, extent of lobar and/or segmental fissure completion, lobar and/or segmental ventilation, lung function, homogeneity/heterogeneity of lobar and/or segmental emphysema, emphysema type, locations of diseased portions of the lung, lobar volume, segmental volume, segmental locations, diaphragm shape, tissue density, opacity, proximity of diseased portions to anatomical structures, proximity of diseased portions to other medical devices, lumen inner diameter of bronchial sections, air flow mapping, collapsed airways, airway pressure, airway compliance, pleural pressure, airway diameter to wall thickness, airway wall deformation, vascular perfusion, parenchyma density, bullae, information that localizes disease in the lung, obstruction score, mucus score, or degree of epithelialization.

Example 8. The method of any one of examples 1-7, wherein the set of lung metrics comprises at least one disease score characterizing severity of pulmonary disease in the patient.

Example 9. The method of example 8, wherein the at least one disease score represents a predictor of patient response to the treatment of the pulmonary disease.

Example 10. The method of example 8 or 9, wherein the set of lung metrics comprises multiple disease scores each corresponding to a respective lobar, segmental, or sub-segmental region of the lung of the patient.

Example 11. The method of example 8 or 9, wherein the set of lung metrics comprises a single disease score based on multiple local disease scores each corresponding to a respective lobar, segmental, or sub-segmental region of the lung of the patient.

Example 12. The method of example 11, wherein the single disease score is an average of the multiple local disease scores.

Example 13. The method of any one of examples 8-12, wherein the at least one disease score represents an extent of at least one of air trapping or hyperinflation in the lung of the patient.

Example 14. The method of any one of examples 7-13, wherein the set of lung metrics characterizes a change in at least one of the one or more lung parameters over a plurality of time points.

Example 15. The method of example 14, wherein the plurality of time points comprise two or more of the following: before endobronchial implant therapy, after endobronchial implant therapy, before administration of a bronchodilator, after administration of a bronchodilator, before exercise, or during exercise.

Example 16. The method of any one of examples 1-15, wherein the predicted response comprises a prediction of one or more of the following: forced expiratory volume in 1 second, forced vital capacity, vital capacity, inspiratory capacity, inspiratory capacity/total lung capacity ratio, functional residual capacity, total lung capacity, diffusion capacity for carbon monoxide, residual volume, residual volume/total lung capacity ratio, segmental volume, mMRC score, SGRQ score or a subset thereof, CAT score or a subset thereof, 6-minute walk test results, cycle ergometry results, cardiopulmonary exercise testing (CPET) results, patient health metrics, patient exercise metrics, patient visit metrics, number of required implant removals, time to reintervention, durability of treatment, quality of life score, body mass index, comorbidities, drug regimen, length of hospitalization, healthcare utilization, or cost.

Example 17. The method of any one of examples 1-16, wherein the treatment comprises an airway treatment for COPD.

Example 18. The method of example 17, wherein the airway treatment comprises a pharmacological treatment.

Example 19. The method of example 17 or 18, wherein the airway treatment comprises an interventional treatment.

Example 20. The method of example 19, wherein the interventional treatment comprises one or more of the following: vapor therapy, administration of a sealant, transbronchial fenestration, placement of an endobronchial coil, placement of an endobronchial valve, or placement of a minimal endobronchial reinforcement implant.

Example 21. The method of example 20, wherein the interventional treatment comprises the placement of the minimal endobronchial reinforcement implant.

Example 22. The method of any one of examples 1-21, further comprising generating a plan for the treatment, if the patient is a candidate for the treatment with the pulmonary disease.

Example 23. The method of example 22, wherein the plan is generated by inputting one or more of the predicted response or the set of lung metrics into a third machine learning algorithm.

Example 24. The method of example 22 or 23, wherein the treatment comprises placement of at least one minimal endobronchial reinforcement implant, and the plan comprises one or more of the following: implant placement location, number of implants, implant size, implant type, pathway to a target location, or localized treatment solutions.

Example 25. The method of example 24, wherein the implant placement location is based at least in part on one of more of the following: location of dynamic airway collapse as determined from expiratory CT data, severity of disease in a peripheral region of the lung, location of a pleural wall of the patient, or location of lobar, segmental, and/or sub-segmental airways.

Example 26. The method of any one of examples 1-25, further comprising generating a report comprising a summary of one or more of the following: at least a portion of the set of lung metrics, the predicted response, the evaluation of whether the patient is a candidate for the treatment, or the generated plan for the treatment.

Example 27. The method of any one of examples 1-26, further comprising updating one or more of the first machine learning algorithm or the second machine learning algorithm based on historical or repository patient data.

Example 28. The method of example 27, wherein the historical or repository patient data comprises data of patients having GOLD III COPD, data of patients having GOLD IV COPD, or a combination thereof.

Example 29. The method of example 27 or 28, wherein the historical or repository patient data comprises data of patients treated with one or more of the following: a minimal endobronchial reinforcement implant, an endobronchial valve, an endobronchial coil, or vapor therapy.

Example 30. The method of any one of examples 27-29, wherein the historical or repository patient data comprises data of the patient from an earlier time point.

Example 31. A system comprising:
a processor; and
a memory operably coupled to the processor and storing instructions that, when executed by the processor, cause the computing system to perform operations comprising the method of any one of examples 1-30.

Example 32. A non-transitory computer-readable storage medium comprising instructions that, when executed by one or more processors of a computing system, cause the computing system to perform operations comprising the method of any one of examples 1-30.

Example 33. A method for evaluating a treatment outcome of a patient, the method comprising:
receiving patient data including computed tomography (CT) data of a lung of the patient after placement of an endobronchial implant in the lung;
generating a set of status metrics by inputting the patient data into a first machine learning algorithm, wherein the set of status metrics includes:
   a set of lung metrics representing a state of the lung of the patient after the placement of the endobronchial implant, and
   a set of implant metrics representing a state of the endobronchial implant after placement in the lung;
determining a response of the patient to the endobronchial implant by inputting the set of status metrics into a second machine learning algorithm; and
predicting an outcome of the patient after the placement of the endobronchial implant by inputting one or more of the set of status metrics or the determined response into a third machine learning algorithm.

Example 34. The method of example 33, wherein the patient data comprises one or more of the following: questionnaire information, medical record information, magnetic resonance imaging (MRI) data, single-photon emission computed tomography (SPECT) data, bronchoscopy data, ventilation-perfusion data, pulmonary function test data, chest radiography data, fluoroscopy data, photographs, or sensor data.

Example 35. The method of example 33 or 34, wherein the CT data comprises expiratory CT data.

Example 36. The method of any one of examples 33-35, wherein the CT data comprises inspiratory CT data.

Example 37. The method of any one of examples 33-36, wherein the patient data comprises data obtained at a plurality of different time points.

Example 38. The method of any one of examples 33-37, wherein the set of lung metrics characterizes one or more lung parameters, the one or more lung parameters characterizing any of the following: forced expiratory volume in 1 second, forced vital capacity, vital capacity, inspiratory capacity, inspiratory capacity/total lung capacity ratio, functional residual capacity, total lung capacity, diffusion capacity for carbon monoxide, residual volume, residual volume/total lung capacity ratio, closing volume, lobar and/or segmental tissue destruction, lobar and/or segmental air trapping, lobar and/or segmental fissure status, extent of lobar and/or segmental fissure completion, lobar and/or segmental ventilation, lung function, homogeneity/heterogeneity of lobar and/or segmental emphysema, emphysema type, location of diseased portions of the lung, lobar volume, segmental volume, segment locations, diaphragm shape, tissue density, opacity, proximity of diseased portions to anatomical structures, proximity of disease portions to other medical devices, lumen inner diameter of bronchial sections, air flow mapping, collapsed airways, airway pressure, airway compliance, pleural pressure, airway diameter to wall thickness, airway wall deformation, vascular perfusion, parenchyma density, bullae, information that localizes disease in the lung, obstruction score, mucus score, degree of epithelialization, granulation tissue, implant-induced airway deformation, or airway tissue invagination into a lumen of the implant.

Example 39. The method of any one of examples 33-38, wherein the set of lung metrics comprises a disease score characterizing severity of pulmonary disease in the patient.

Example 40. The method of example 38 or 39, wherein the set of lung metrics characterizes a change in at least one of the one or more lung parameters over a plurality of time points.

Example 41. The method of example 40, wherein the plurality of time points comprise two or more of the following: before endobronchial implant therapy, after endobronchial implant therapy, before administration of a bronchodilator, after administration of a bronchodilator, before exercise, or during exercise.

Example 42. The method of any one of examples 33-41, wherein the endobronchial implant comprises a minimal endobronchial reinforcement implant.

Example 43. The method of any one of examples 33-42, wherein the set of implant metrics characterizes one or more of the following: implant location, distance between a distal end of the implant and pleura, implant length, implant diameter at any one or more locations along a length of the implant, implant cross-sectional profile at any one or more locations along a length of the implant, implant integrity, pitch of loops of an implant, angle of an implant loop profile relative to a longitudinal axis of the implant, implant position relative to one or more additional implants, movement of the implant between inspiration and expiration, occlusion of the implant, or implant dislodgment.

Example 44. The method of any one of examples 33-43, further comprising generating and displaying a virtual bronchoscopy depicting a model incorporating one or more of at least a portion of the lung metrics or at least a portion of the implant metrics.

Example 45. The method of any one of examples 33-44, wherein the determined response comprises a determination of one or more of the following: forced expiratory volume in 1 second, forced vital capacity, vital capacity, inspiratory capacity, inspiratory capacity/total lung capacity ratio, functional residual capacity, total lung capacity, diffusion capacity for carbon monoxide, residual volume, residual volume/total lung capacity ratio, segmental volume, mMRC score, SGRQ score or a subset thereof, CAT score or a subset thereof, 6-minute walk test results, cycle ergometry results, cardiopulmonary exercise testing (CPET) results, patient health metrics, patient exercise metrics, patient visit metrics, number of required implant removals, time to reintervention, durability of treatment, quality of life score, body mass index, comorbidities, drug regimen, length of hospitalization, healthcare utilization, or cost.

Example 46. The method of any one of examples 33-45, wherein the predicted outcome comprises a prediction of a post-procedure issue after the placement of the endobronchial implant.

Example 47. The method of example 46, wherein the post-procedure issue comprises one or more of the following: copious mucus, excessive granulation tissue, excessive fibrosis, implant collapse, implant failure, implant migration, implant expectoration, inadequate lung function, pneumothorax, infection, pneumonia, or hospitalization.

Example 48. The method of example 46 or 47, further comprising determining an intervention to address the post-procedure issue.

Example 49. The method of example 48, wherein the determined intervention comprises one or more of the following: cleanup bronchoscopy, retrieval or removal of the endobronchial implant, repositioning of the endobronchial implant, replacement of the endobronchial implant, dilation of the endobronchial implant, placement of an additional endobronchial implant, or consultation with a healthcare professional.

Example 50. The method of any one of examples 33-49, further comprising generating a report comprising a summary of one or more of the following: at least a portion of the lung metrics, at least a portion of the implant metrics, the determined response of the patient to the endobronchial implant, the predicted outcome of the patient after the placement of the endobronchial implant, or the determined intervention to address a post-procedure issue.

Example 51. The method of any one of examples 33-50, further comprising updating one or more of the first machine learning algorithm, the second machine learning algorithm, or the third machine learning algorithm based on historical or repository patient data.

Example 52. The method of example 51, wherein the historical or repository patient data comprises data of patients having GOLD III COPD, data of patients having GOLD IV COPD, or a combination thereof.

Example 53. The method of example 51 or 52, wherein the historical or repository patient data comprises data of patients treated with one or more of the following: a minimal endobronchial reinforcement implant, an endobronchial valve, an endobronchial coil, or vapor therapy.

Example 54. The method of any one of examples 51-53, wherein the historical or repository patient data comprises data of the patient from an earlier time point.

Example 55. The method of any one of examples 33-54, further comprising comparing the set of lung metrics to a set of second lung metrics determined from one or more of the following: image data of the lung before the placement of the endobronchial implant, image data of the lung at an earlier time point after the placement of the endobronchial implant, image data of the lung after placement of another endobronchial implant at a different location than a location of the endobronchial implant, or image data from other patients having COPD.

Example 56. A system comprising:
a processor; and
a memory operably coupled to the processor and storing instructions that, when executed by the processor, cause the computing system to perform operations comprising the method of any one of examples 33-55.

Example 57. A non-transitory computer-readable storage medium comprising instructions that, when executed by one or more processors of a computing system, cause the computing system to perform operations comprising the method of any one of examples 33-55.

Example 58. A method for evaluating a patient having or suspected of having a pulmonary disease, the method comprising:
receiving patient data including computed tomography (CT) data of a lung of the patient; and
generating a pulmonary disease score for a region of interest of the lung by inputting the patient data into a machine learning algorithm, wherein the pulmonary disease score characterizes a severity of pulmonary disease in the region of interest of the lung of the patient, wherein the region of interest is a segmental region or a sub-segmental region of the lung.

Example 59. The method of example 58, wherein the machine learning algorithm evaluates voxel density in the CT data associated with the region of interest of the lung of the patient.

Example 60. The method of example 58 or 59, wherein the pulmonary disease score is based on multiple local disease scores each corresponding to a respective lobar, segmental, or sub-segmental region of the lung of the patient.

Example 61. The method of example 60, wherein the pulmonary disease score is an average of the multiple local disease scores.

Example 62. The method of example 58 or 59, wherein the pulmonary disease score is a first pulmonary disease score, wherein the method further comprises generating a plurality of pulmonary disease scores comprising the first pulmonary disease score, wherein each of the plurality of pulmonary disease scores corresponds to a respective lobar, segmental, or sub-segmental region of the lung of the patient.

Example 63. The method of any one of examples 58-62, wherein the pulmonary disease score represents an extent of at least one of air trapping or hyperinflation in the lung of the patient.

Example 64. The method of any one of examples 58-63, wherein the CT data comprises expiratory CT data.

Example 65. The method of any one of examples 58-64, wherein the CT data comprises inspiratory CT data.

Example 66. The method of any one of examples 58-65, wherein the CT data is generated prior to a treatment administered to the patient to treat the pulmonary disease.

Example 67. The method of any one of examples 58-65, wherein the CT data is generated following a treatment administered to the patient to treat the pulmonary disease.

Example 68. The method of example 66 or 67, wherein the treatment comprises placement of an endobronchial implant.

Example 69. A system comprising:
a processor; and
a memory operably coupled to the processor and storing instructions that, when executed by the processor, cause the computing system to perform operations comprising the method of any one of examples 58-68.

Example 70. A computed tomography (CT) scanner comprising:
a processor; and
a memory operably coupled to the processor and storing instructions that, when executed by the processor, cause the CT scanner to perform operations comprising the method of any one of examples 58-68.

Example 71. A non-transitory computer-readable storage medium comprising instructions that, when executed by one or more processors of a computing system, cause the computing system to perform operations comprising the method of any one of examples 58-68.

Example 72. A method for normalizing quantitative computed tomography (CT) results for a patient, the method comprising:
receiving first CT data for the patient, wherein the first CT data is generated under predetermined imaging conditions; and
transforming the first CT data to second CT data by applying to the first CT data at least one correction factor associated with the predetermined imaging conditions.

Example 73. The method of example 72, wherein the at least one correction factor maps voxel density in the first CT data to a normalized voxel density.

Example 74. The method of example 72 or 73, wherein the at least one correction factor compensates for voxel density in the first CT data affected by one or more of the following: tube current, tube potential, pitch,

Example 75. The method of any one of examples 72-74, wherein the at least one correction factor compensates for voxel density in the first CT data affected by at least one of slice thickness or slice interval.

Example 76. The method of any one of examples 72-75, wherein the at least one correction factor compensates for voxel density in the first CT data affected by a reconstruction algorithm for determining sharpness or smoothness of image in an axial plane.

Example 77. The method of any one of examples 72-76, wherein the first CT data is obtained from a CT scan provider having a provider-specific machine learning algorithm for reconstructing a CT image from CT data, wherein the at least one correction factor compensates for voxel density in the first CT data affected by the provider-specific machine learning algorithm.

Example 78. The method of any one of examples 72-77, wherein the at least one correction factor compensates for voxel density in the first CT data affected by administration of a contrast agent in the patient before the first CT data is generated.

Example 79. The method of any one of examples 72-78, wherein the second CT data is normalized with respect to CT scan parameters.

Example 80. The method of any one of examples 72-79, wherein the first CT data is obtained during a pre-procedure phase prior to placement of an endobronchial implant in the patient.

Example 81. The method of any one of examples 72-80, further comprising generating a set of lung metrics associated with the patient based on the second CT data.

Example 82. The method of any one of examples 72-79, wherein the first CT data is obtained during a peri-procedure phase during placement of an endobronchial implant in the patient.

Example 83. The method of any one of examples 72-79, wherein the first CT data is obtained during a post-procedure phase following placement of an endobronchial implant in the patient.

Example 84. The method of example 82 or 83, further comprising generating at least one of a set of lung metrics or a set of implant metrics associated with the patient based on the second CT data.

Example 85. A system comprising:
a processor; and
a memory operably coupled to the processor and storing instructions that, when executed by the processor, cause the computing system to perform operations comprising the method of any one of examples 72-84.

Example 86. A computed tomography (CT) scanner comprising:
a processor; and
a memory operably coupled to the processor and storing instructions that, when executed by the processor, cause the CT scanner to perform operations comprising the method of any one of examples 72-84.

Example 87. A non-transitory computer-readable storage medium comprising instructions that, when executed by one or more processors of a computing system, cause the computing system to perform operations comprising the method of any one of examples 72-84.

Example 88. A method for planning a treatment for a patient having a pulmonary disease, the method comprising:
receiving patient data including computed tomography (CT) data of a lung of the patient;
generating a set of lung metrics by inputting the patient data into a first machine learning algorithm, wherein the set of lung metrics represents a state of the lung of the patient;
identifying a potential target region in the lung for a treatment for the pulmonary disease, based at least in part on the generated lung metrics.

Example 89. The method of example 88, wherein the patient data comprises one or more of the following: questionnaire information, medical record information, magnetic resonance imaging (MRI) data, single-photon emission computed tomography (SPECT) data, bronchoscopy data, ventilation-perfusion data, pulmonary function test data, chest radiography data, fluoroscopy data, photographs, or sensor data.

Example 90. The method of example 88 or 89, wherein the CT data comprises expiratory CT data.

Example 91. The method of any one of examples 88-90, wherein the CT data comprises inspiratory CT data.

Example 92. The method of any one of examples 88-91, wherein the patient data comprises data obtained at a plurality of different time points.

Example 93. The method of any one of examples 88-92, wherein the set of lung metrics correlates to whether the patient has at least one of chronic obstructive pulmonary disease (COPD), severe emphysema, or severe emphysema with hyperinflation.

Example 94. The method of any one of examples 88-93, wherein the set of lung metrics characterizes one or more lung parameters, the one or more lung parameters comprising one or more of the following: forced expiratory volume in 1 second, forced vital capacity, vital capacity, inspiratory capacity, inspiratory capacity/total lung capacity ratio, functional residual capacity, total lung capacity, diffusion capacity for carbon monoxide, residual volume, residual volume/total lung capacity ratio, closing volume, lobar and/or segmental tissue destruction, lobar and/or segmental air trapping, lobar and/or segmental fissure status, extent of lobar and/or segmental fissure completion, lobar and/or segmental ventilation, lung function, homogeneity/heterogeneity of lobar and/or segmental emphysema, emphysema type, locations of diseased portions of the lung, lobar volume, segmental volume, segmental locations, diaphragm shape, tissue density, opacity, proximity of diseased portions to anatomical structures, proximity of diseased portions to other medical devices, lumen inner diameter of bronchial sections, air flow mapping, collapsed airways, airway pressure, airway compliance, pleural pressure, airway diameter to wall thickness, airway wall deformation, vascular perfusion, parenchyma density, bullae, information that localizes disease in the lung, obstruction score, mucus score, or degree of epithelialization.

Example 95. The method of any one of examples 88-94, wherein the set of lung metrics comprises at least one disease score characterizing severity of pulmonary disease in the patient.

Example 96. The method of example 95, wherein the at least one disease score represents a predictor of patient response to the treatment of the pulmonary disease.

Example 97. The method of example 95 or 96, wherein the set of lung metrics comprises multiple disease scores each corresponding to a respective lobar, segmental, or sub-segmental region of the lung of the patient.

Example 98. The method of example 95 or 96, wherein the set of lung metrics comprises a single disease score based on multiple local disease scores each corresponding to a respective lobar, segmental, or sub-segmental region of the lung of the patient.

Example 99. The method of example 98, wherein the single disease score is an average of the multiple local disease scores.

Example 100. The method of any one of examples 95-99, wherein the at least one disease score represents an extent of at least one of air trapping or hyperinflation in the lung of the patient.

Example 101. The method of any one of examples 94-100, wherein the set of lung metrics characterizes a change in at least one of the one or more lung parameters over a plurality of time points.

Example 102. The method of example 101, wherein the plurality of time points comprise two or more of the following: before endobronchial implant therapy, after endobronchial implant therapy, before administration of a bronchodilator, after administration of a bronchodilator, before exercise, or during exercise.

Example 103. The method of any one of examples 88-102, wherein the treatment comprises an airway treatment for COPD.

Example 104. The method of example 103, wherein the airway treatment comprises a pharmacological treatment.

Example 105. The method of example 103 or 104, wherein the airway treatment comprises an interventional treatment.

Example 106. The method of example 105, wherein the interventional treatment comprises one or more of the following: vapor therapy, administration of a sealant, transbronchial fenestration, placement of an endobronchial coil, placement of an endobronchial valve, or placement of a minimal endobronchial reinforcement implant.

Example 107. The method of example 106, wherein the interventional treatment comprises the placement of the minimal endobronchial reinforcement implant.

Example 108. The method of any one of examples 88-107, further comprising generating a plan for the treatment.

Example 109. The method of example 108, wherein the plan is generated by inputting the set of lung metrics into a second machine learning algorithm.

Example 110. The method of example 108 or 109, wherein the treatment comprises placement of at least one minimal endobronchial reinforcement implant, and the plan comprises one or more of the following: implant placement location, number of implants, implant size, implant type, pathway to a target location, or localized treatment solutions.

Example 111. The method of example 110, wherein the implant placement location is based at least in part on one of more of the following: location of dynamic airway collapse as determined from expiratory CT data, severity of disease in a peripheral region of the lung, location of a pleural wall of the patient, or location of lobar, segmental, and/or sub-segmental airways.

Example 112. A system comprising:
a processor; and
a memory operably coupled to the processor and storing instructions that, when executed by the processor, cause the computing system to perform operations comprising the method of any one of examples 88-111.

Example 113. A computed tomography (CT) scanner comprising:
a processor; and
a memory operably coupled to the processor and storing instructions that, when executed by the processor, cause the CT scanner to perform operations comprising the method of any one of examples 88-111.

Example 114. A non-transitory computer-readable storage medium comprising instructions that, when executed by one or more processors of a computing system, cause the computing system to perform operations comprising the method of any one of examples 88-111.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIG. 1 is a schematic illustration of a bronchial tree of a human subject within a chest cavity of the subject.

FIG. 2 is a schematic illustration of a bronchial tree of a human subject in isolation.

FIG. 3 is an enlarged view of a terminal portion of the bronchial tree shown in FIG. 2.

FIG. 4 is a table showing examples of dimensions and generation numbers of different portions of a bronchial tree of a human subject.

FIG. 5 is a diagram showing lung volumes during normal lung function.

FIG. 6 is a table showing airway wall composition at different portions of a bronchial tree of a human subject.

FIG. 7 is an anatomical illustration of airway wall composition at different portions of a bronchial tree of a human subject.

FIG. 8 is an anatomical illustration showing small airway narrowing in emphysematous lung tissue.

FIG. 9 is an anatomical illustration showing alveolar wall damage in emphysematous lung tissue.

FIG. 10 is an anatomical illustration showing normal airway patency during exhalation in healthy lung tissue.

FIG. 11 is an anatomical illustration showing airway collapse during exhalation in emphysematous lung tissue.

FIG. 12 is an anatomical illustration showing normal acinar.

FIG. 13 is an anatomical illustration showing centriacinar emphysema.

FIG. 14 is an anatomical illustration showing panacinar emphysema.

FIG. 15 is an anatomical illustration showing paraseptal emphysema.

FIG. 16 is a side view of an implant in accordance with at least some embodiments of the present technology.

FIG. 17 is a schematic end view of the implant shown in FIG. 16.

FIG. 18 is a side view of a portion of an implant in accordance with at least some embodiments of the present technology within an airway.

FIG. 19 is a block diagram providing a general overview of a workflow for the selection of patients for treatment, and planning and monitoring a treatment procedure, in accordance with embodiments of the present technology.

FIG. 20 is a flow diagram illustrating a method for planning a treatment for a patient, in accordance with embodiments of the present technology.

FIG. 21 is a flow diagram illustrating a method for evaluating a treatment outcome of a patient, in accordance with embodiments of the present technology.

FIG. 22 is a flow diagram illustrating a method for updating the software algorithms of FIGS. 20 and 21, in accordance with embodiments of the present technology.

FIGS. 23A and 24B are flow diagrams illustrating examples of generating various lung metrics based on inspiratory CT scans and expiratory CT scans, respectively.

FIG. 24 is a flow diagram illustrating an example of assessing treatment effect and/or efficacy based on a CT scan.

FIG. 25A is an anatomical illustration showing a coronal view of fissures in the right and left lungs. FIG. 25B is an anatomical illustration showing a sagittal view of fissures in the right lung. FIG. 25C is an anatomical illustration showing a sagittal view of fissures in the left lung.

FIG. 26 is a flow diagram illustrating a method for normalizing quantitative CT results.

FIG. 27 is a flow diagram illustrating a method for evaluating a patient having or suspected of having a pulmonary disease.

FIG. 28 is a flow diagram illustrating a method for planning a treatment for a patient having a pulmonary disease.

### DETAILED DESCRIPTION

The present technology relates to methods for planning, predicting, and/or monitoring treatment procedures for patients having a pulmonary disease, such as COPD. In some embodiments, for example, a method for planning a treatment for a patient includes receiving patient data including computed tomography (CT) data of a lung of the patient. The method can include generating a set of lung metrics by inputting the patient data into a first machine learning algorithm. The method can include predicting a response of the patient to treatment for the pulmonary disease (e.g., treatment with an endobronchial implant) by inputting the set of lung metrics into a second machine learning algorithm. The method can further include evaluating whether the patient is a candidate for the treatment for the pulmonary disease, based on the predicted response.

As another example, a method for evaluating a treatment outcome of a patient includes receiving patient data including CT data of a lung of the patient after placement of an endobronchial implant in the lung. The method can include generating a set of status metrics (e.g., lung metrics, implant metrics) by inputting the patient data into a first machine learning algorithm. The method can also include determining a response of the patient to the endobronchial implant by inputting the set of status metrics into a second machine learning algorithm. The method can further include predicting an outcome of the patient after the placement of the endobronchial implant by inputting the set of status metrics and/or the determined response into a third machine learning algorithm.

The present technology can provide numerous advantages for treatment of patients with pulmonary disease. For instance, the methods described herein can be used to diagnose and treat patients at an earlier stage of the disease (e.g., stage 2 COPD), which can improve therapeutic efficacy and lead to better outcomes. Additionally, the methods herein can provide patients with personalized, evidence-based treatment recommendations that are more likely to lead to successful results. The methods herein can also improve planning of treatment procedures, which can reduce procedure time, improve patient safety, and lead to improved outcomes. Moreover, the methods of the present technology can monitor the patient after the procedure to predict problems before they occur, thus reducing the frequency of additional hospitalization and doctor visits after the treatment procedure.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The headings provided herein are for convenience only and do not interpret the scope or meaning of the claimed present technology. Embodiments under any one heading may be used in conjunction with embodiments under any other heading.

### I. Anatomy and Physiology

In normal respiration, the act of inhaling draws air into the lungs via the nose or mouth and the trachea. Within each lung, inhaled air moves into a branching network of progressively narrower airways called bronchi, and then into the narrowest airways called bronchioles. The bronchioles end in bunches of tiny round structures called alveoli. Small blood vessels called capillaries run through the walls of the alveoli. When inhaled air reaches the alveoli, oxygen moves from the alveoli into blood in the capillaries. At the same time, carbon dioxide moves in the opposite direction, i.e., from blood in the capillaries into the alveoli. This process is called gas exchange. In a healthy lung, the airways and alveoli are elastic and stretch to accommodate air intake. When a breath is drawn in, the alveoli fill up with air like small balloons. When a breath is expelled, the alveoli deflate. This expansion of the alveoli is an important part of effective gas exchange. Alveoli that are free to expand exchange more gas than alveoli that are inhibited from expanding.

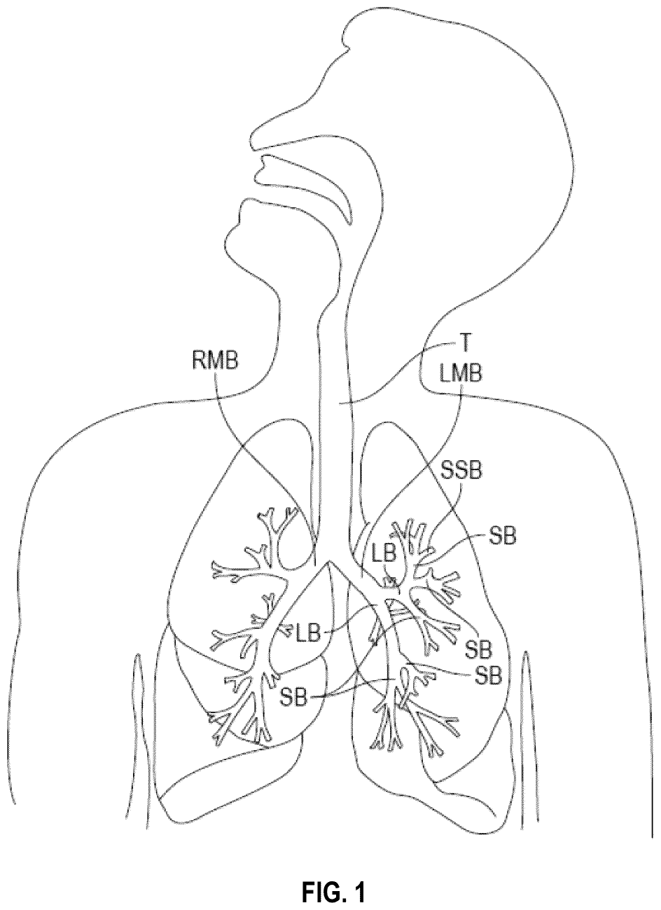

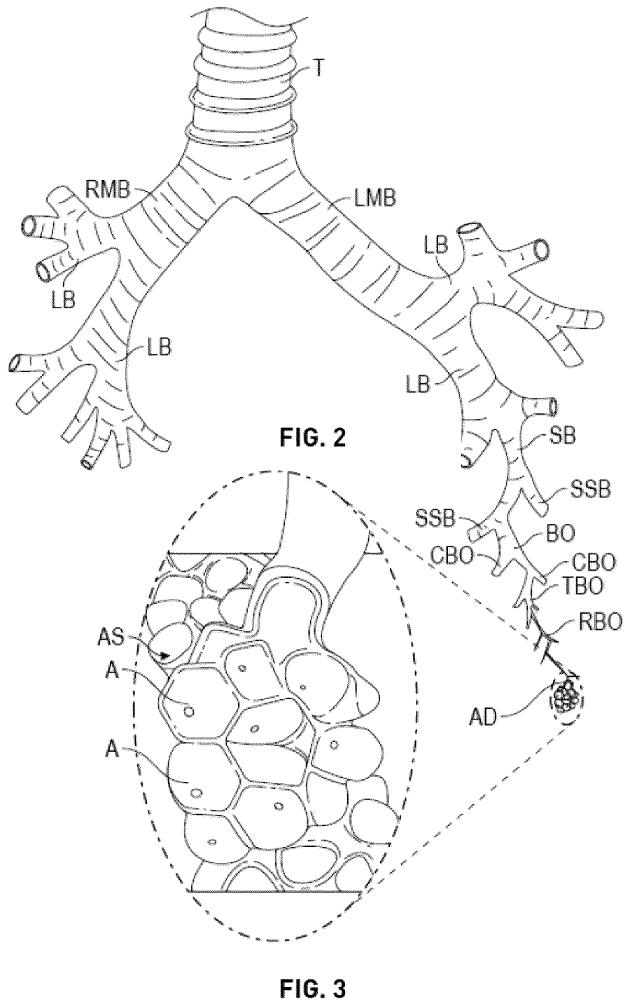

FIG. 1 is a schematic illustration of a bronchial tree of a human subject within a chest cavity of the subject. As shown in FIG. 1, the bronchial tree includes a trachea T that extends downwardly from the nose and mouth and divides into a left main bronchus LMB and a right main bronchus RMB. The left main bronchus and the right main bronchus each branch to form lobar bronchi LB, segmental bronchi SB, and sub-segmental bronchi SSB, which have successively smaller diameters and shorter lengths as they extend distally. FIG. 2 is a schematic illustration of the bronchial tree in isolation. As shown in FIG. 2, the sub-segmental bronchi continue to branch to form bronchioles BO, conducting bronchioles CBO, and finally terminal bronchioles TBO, which are the smallest airways that do not contain alveoli. The terminal bronchioles branch into respiratory bronchioles RBO, which divide into alveolar ducts AD. FIG. 3 is an enlarged view of a terminal portion of the bronchial tree. As shown in FIG. 3, the alveolar ducts terminate in a blind outpouching including two or more small clusters of alveoli A called alveolar sacs AS. Various singular alveoli can be disposed along the length of a respiratory bronchiole as well.

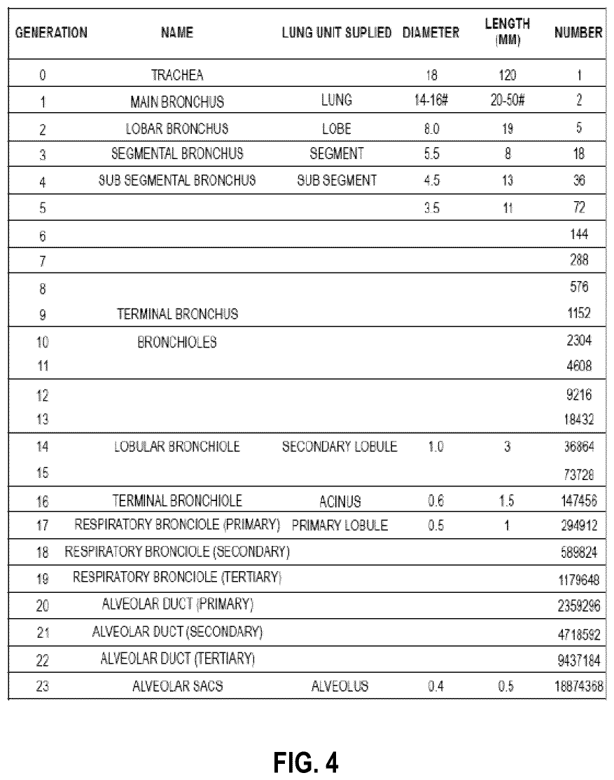

Bronchi and bronchioles are conducting airways that convey air to and from the alveoli. They do not take part in gas exchange. Rather, gas exchange takes place in the alveoli that are found distal to the conducting airways, starting at the respiratory bronchioles. It is common to refer to the various airways of the bronchial tree as “generations” depending on the extent of branching proximal to the airways. For example, the trachea is referred to as “generation 0” of the bronchial tree, various levels of bronchi, including the left and right main bronchi, are referred to as “generation 1,” the lobar bronchi are referred to as “generation 2,” and the segmental bronchi are referred to as “generation 3.” Further, it is common to refer to any of the airways extending from the trachea to the terminal bronchioles as “conducting airways.” FIG. 4 is a table indicating examples of dimensions and generation numbers of different portions of the bronchial tree.

The respiratory bronchioles, alveoli, and alveolar sacs receive air via more proximal portions of the bronchial tree and participate in gas exchange to oxygenate blood routed to the lungs from the heart via the pulmonary artery, branching blood vessels, and capillaries. Thin, semi-permeable membranes separate oxygen-depleted blood in the capillaries from oxygen-rich air in the alveoli. The capillaries wrap around and extend between the alveoli. Oxygen from the air diffuses through the membranes into the blood. Carbon dioxide from the blood diffuses through the membranes to the air in the alveoli. The newly oxygen-enriched blood then flows from the alveolar capillaries through the branching blood vessels of the pulmonary venous system to the heart. The heart pumps the oxygen-rich blood throughout the body. The oxygen-depleted air in the lungs is exhaled when the diaphragm and intercostal muscles relax and the lungs and chest wall elastically return to their normal relaxed states. In this manner, air flows through the branching bronchioles, segmental bronchi, lobar bronchi, main bronchi, and trachea, and is ultimately expelled through the mouth and nose.

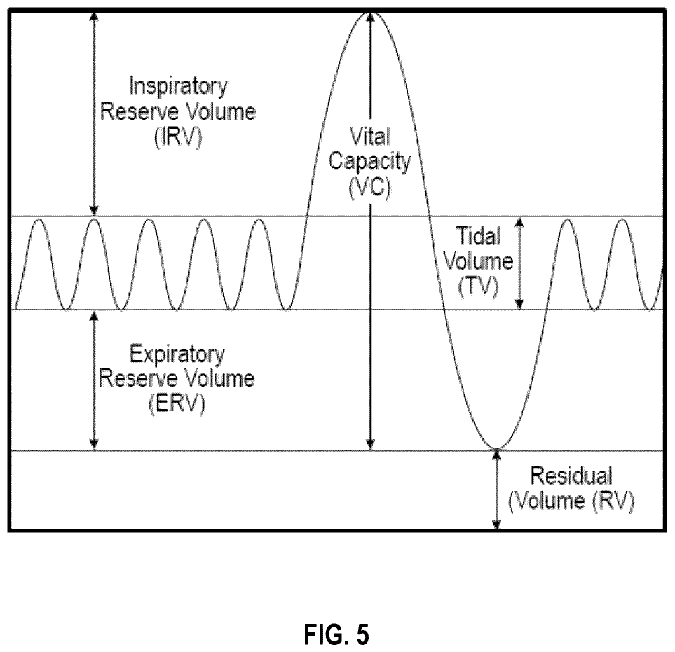

FIG. 5 is a diagram showing lung volumes during normal lung function. Approximately one-tenth of the total lung capacity is used at rest. Greater amounts are used as needed (e.g., with exercise). Tidal Volume (TV) is the volume of air breathed in and out without conscious effort. The additional volume of air that can be exhaled with maximum effort after a normal inspiration is Inspiratory Reserve Volume (IRV). The additional volume of air that can be forcibly exhaled after normal exhalation is Expiratory Reserve Volume (ERV). The total volume of air that can be exhaled after a maximum inhalation is Vital Capacity (VC). VC equals the sum of the TV, IRV, and ERV. Residual Volume (RV) is the volume of air remaining in the lungs after maximum exhalation. The lungs can never be completely emptied. The Total Lung Capacity (TLC) is the sum of the VC and RV. Evaluation of lung function may be used to determine a patient's eligibility for therapy, as well as to evaluate a therapy's effectiveness.

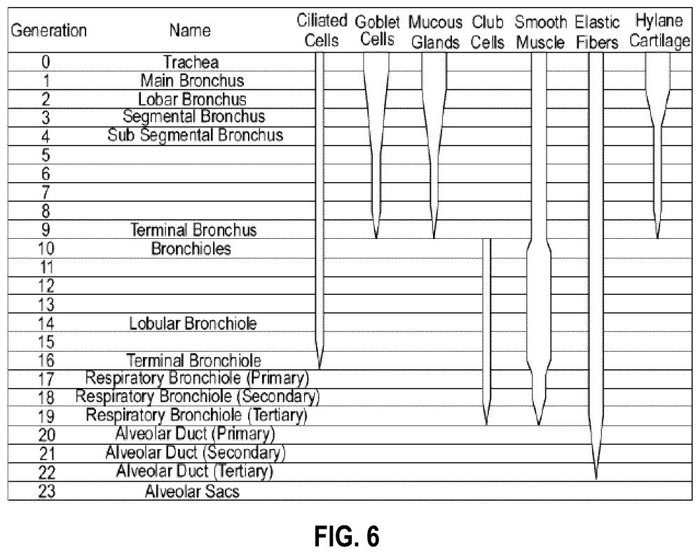

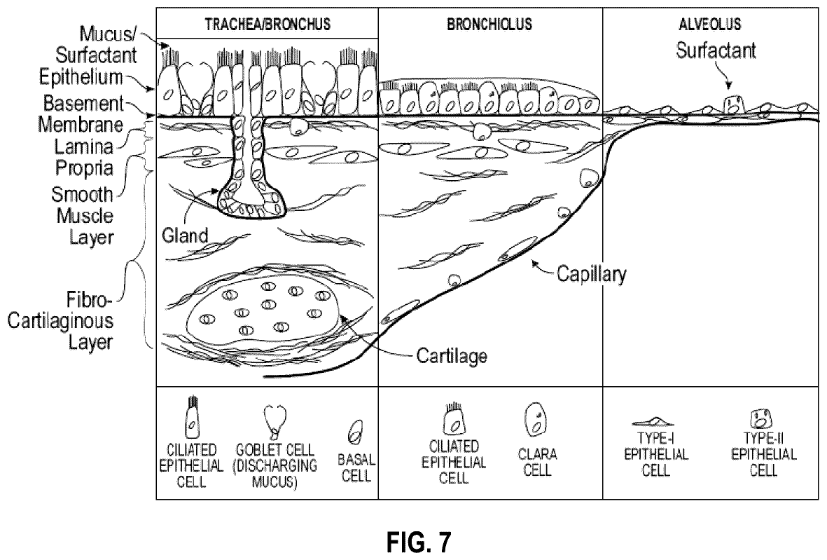

FIG. 6 is a table showing airway wall composition at different portions of a bronchial tree. FIG. 7 is an anatomical illustration of airway wall composition at different portions of a bronchial tree. As shown in FIGS. 6 and 7, the walls of the bronchi, bronchioles, alveolar ducts and alveoli include epithelium, connective tissue, goblet cells, mucous glands, club cells, smooth muscle elastic fibers, and hyaline cartilage with nerves, blood vessels, and inflammatory cells interspersed throughout. Most of the epithelium (from the nose to the bronchi) is covered in ciliated pseudostratified columnar epithelium, commonly called respiratory epithelium. The cilia located on these epithelium beat in one direction, moving mucous and foreign material such as dust and bacteria from the more distal airways to the more proximal airways and eventually to the throat, where the mucus and/or foreign material are cleared by swallowing or expectoration. Moving down the bronchioles, the cells are more cuboidal in shape but are still ciliated.

The proportions and properties of various components of the airway wall vary depending on the location within the bronchial tree. For example, mucous glands are abundant in the trachea and main bronchi but are absent starting at the bronchioles (e.g., at approximately generation 10). In the trachea, cartilage presents as C-shaped rings of hyaline cartilage, whereas in the bronchi the cartilage takes the form of interspersed plates. As branching continues through the bronchial tree, the amount of hyaline cartilage in the walls decreases until it is absent in the bronchioles. Smooth muscle starts in the trachea, where it joins the C-shaped rings of cartilage. It continues down the bronchi and bronchioles, which it completely encircles. Instead of hard cartilage, the bronchi and bronchioles are composed of elastic tissue. As the cartilage decreases, the amount of smooth muscle increases. The mucous membrane also undergoes a transition from ciliated pseudostratified columnar epithelium to simple cuboidal epithelium to simple squamous epithelium.

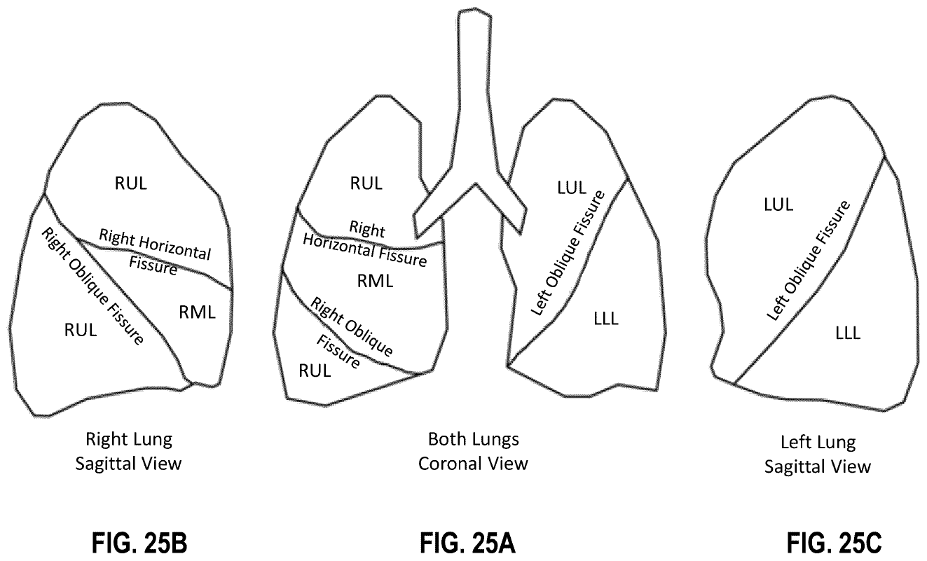

FIGS. 25A-25C are anatomical illustrations of fissures of the right and left lungs. The right horizontal fissure separates the right upper lobe (RUL) and the right middle lobe (RML). The right oblique fissure divides the right middle lobe (RML) and the right lower lobe (RLL), and separates the RUL and the RLL posteriorly. The left oblique fissure separates the left upper lobe (LUL) and the left lower lobe (LLL). In normal, healthy tissue, pulmonary fissures are double layers of infolded invaginations of visceral pleura, and exit between the different lobes. Additionally, there are segmental fissures (not shown) separating 18 segments across the five lobes (RUL, RML, RLL, LUL, LLL). However, the appearance of fissures may vary widely from patient to patient, and may be incomplete or even absent and/or distorted (in position, shape, etc.) due to diseases such as COPD. Integrity or degree of completeness of fissures indicates how well-separated the lung lobes are. Incomplete fissures may, for example, indicate that air from one lobe can flow into another (e.g., collateral ventilation). CT imaging may be used to visualize certain features of lung fissures, though different CT protocols may lead to different appearances of the fissure. One or more various algorithms may be used to automatically identify and/or characterize lung fissures in CT imaging, such as fissure segmentation algorithms (e.g., an algorithm to perform implicit surface fitting to a surface shaped structure of the lung volume, a trained machine learning algorithm such as a supervised fissure enhancement filter, an algorithm to perform adaptive fissure sweeping and wavelet transform, etc.) and/or algorithms based on anatomical knowledge (e.g., fuzzy reasoning system to search fissures based at least in part on ridgeness image intensity and smoothness, etc.), and/or other suitable algorithms for fissure characterization.

### II. Pulmonary Disease

COPD is a major public health issue. There are over one million patients in the United States alone with severe emphysema and severe hyperinflation. An overwhelming majority of these patients are underserved by currently available treatments. The global unmet clinical need, including in countries with high incidence of respiratory disease due to smoking, is many times greater than in the United States.

In COPD-affected lung tissue, less air flows through the airways for a variety of reasons. The airways and/or alveoli may be relatively inelastic, the walls between the alveoli may be damaged or destroyed, the walls of the airways may be thick or inflamed, and/or the airways may generate excessive mucus resulting in mucus buildup and airway blockage. In a typical case of COPD, the disease does not equally affect all airways and alveoli in a lung. A lung may have some regions that are significantly more affected than other regions. In severe cases, the airways and alveoli that are unsuitable for effective gas exchange may make up 20 to 30 percent or more of total lung volume.

The effects of COPD are often most pronounced when a patient exercises or engages in other physical exertion that would cause a healthy person to breath heavily. A patient with COPD may not be able to breathe heavily because diseased portions of the patient's lungs trap air, resulting in an inability to exhale completely. This, in turn, inhibits subsequent expansion of healthy lung tissue. Thus, during exercise or other physical exertion, the lungs of a COPD patient may operate in a state of dynamic hyperinflation that impairs respiratory mechanics and increases the work of breathing. Hyperinflation of the lungs may also hinder cardiac filling, lead to dyspnea, and/or reduce a patent's exercise performance. These and/or other detrimental effects of COPD can lead to a cascade of symptoms that eventually impairs a patient's quality of life and increases the risk of severe disability and death.

The term COPD includes both chronic bronchitis and emphysema. About 25% of COPD patients have emphysema. About 40% of these emphysema patients have severe emphysema. Furthermore, it is common for COPD patients to have symptoms of both chronic bronchitis and emphysema. In chronic bronchitis, the lining of the airways is inflamed, generally as a result of ongoing irritation. This inflammation results in thickening of the airway lining and in production of a thick mucus that may coat and eventually congest the airways. Emphysema, in contrast, is primarily a pathological diagnosis concerning abnormal permanent enlargement of air spaces distal to the terminal bronchioles. In emphysematous lung tissue, the small airways and/or alveoli typically have lost their structural integrity and/or their ability to maintain an optimal shape. For example, damage to or destruction of alveolar walls may have resulted in fewer, but larger alveoli. This may significantly impair normal gas exchange. Within the lung, focal or “diseased” regions of emphysematous lung tissue characterized by a lack of discernible alveolar walls may be referred to as pulmonary bullae. These relatively inelastic pockets of dead space are often greater than 1 cm in diameter and do not contribute significantly to gas exchange. Pulmonary bullae tend to retain air and thereby create hyperinflated lung sections that restrict the ability of healthy lung tissue to fully expand upon inhalation. Accordingly, in patients with emphysema, not only does the diseased lung tissue no longer contribute significantly to respiratory function, it impairs the functioning of healthy lung tissue.

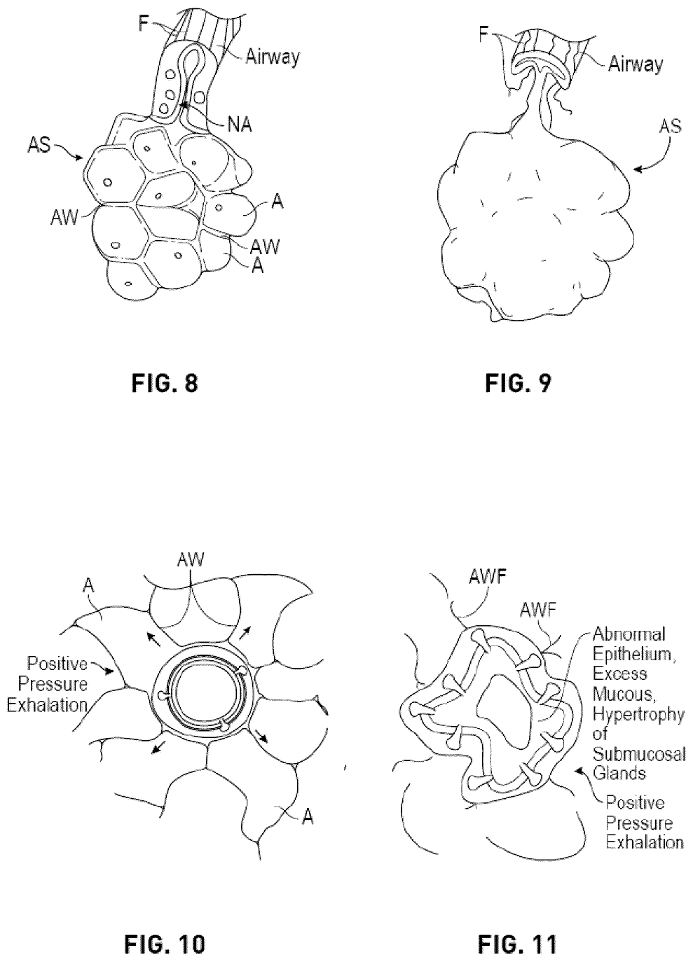

FIG. 8 is an anatomical illustration showing small airway narrowing in emphysematous lung tissue. FIG. 9 is an anatomical illustration showing alveolar wall damage in emphysematous lung tissue. FIG. 10 is an anatomical illustration showing normal airway patency during exhalation. FIG. 11 is an anatomical illustration showing airway collapse during exhalation in emphysematous lung tissue. COPD, and emphysema in particular, is characterized by irreversible destruction of the alveolar walls that contain elastic fibers that maintain radial outward traction on small airways and are useful in inhalation and exhalation. As shown in FIGS. 8-11, when these elastic fibers are damaged, the small airways are no longer under radial outward traction and collapse, particularly during exhalation. Furthermore, emphysema destroys the alveolar walls. As shown in FIG. 9, this results in one larger air space and reduces the surface area available for gas exchange. The lungs are thus unable to perform gas exchange at a satisfactory rate, which causes a reduction in oxygenated blood. Additionally, the large air spaces of diseased lung combined with collapsed airways results in hyperinflation (air trapping) of the lung and an inability to fully exhale. Moreover, the hyperinflated lungs apply continuous pressure on the chest wall, diaphragm, and surrounding structures, which causes shortness of breath and can prevent a patient from walking short distances or performing routine tasks. Both quality of life and life expectancy for patients with late-stage emphysema are extremely low, with fewer than half of patients surviving an additional five years.

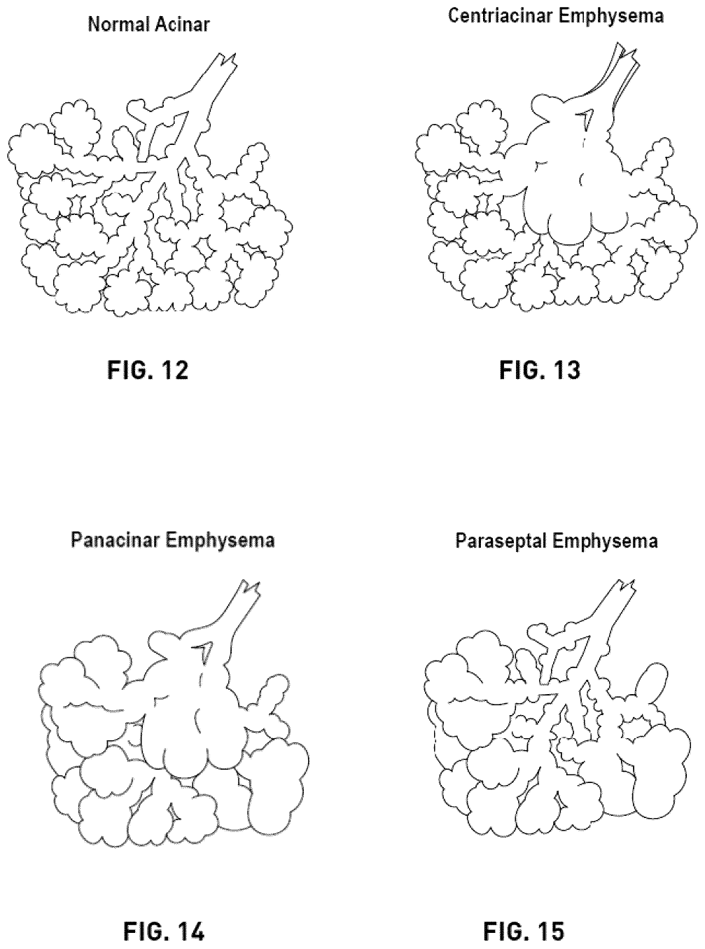

There are three types of emphysema: centriacinar, panacinar, and paraseptal. FIG. 12 is an anatomical illustration showing normal acinar. FIG. 13 is an anatomical illustration showing centriacinar emphysema, which involves the alveoli and airways in the central acinus, including destruction of the alveoli in the walls of the respiratory bronchioles and alveolar ducts. FIG. 14 is an anatomical illustration showing panacinar emphysema, which is characterized by destruction of the tissues of the alveoli, alveolar ducts, and respiratory bronchioles. This produces a fairly uniform dilatation of the air space throughout the acini and evenly distributed emphysematous changes across the acini and the secondary lobules. FIG. 15 is an anatomical illustration showing paraseptal emphysema, which is characterized by enlarged airspaces at the periphery of acini resulting predominately from destruction of the alveoli and alveolar ducts. The distribution of the paraseptal emphysema is usually limited in extent and occurs most commonly along the posterior surface of the upper lung. It often coexists with other forms of emphysema.

Emphysema can also be characterized as heterogenous or homogenous. Generally, heterogenous emphysema in a lung (right or left) is characterized by any two or more regions (e.g., lobes, segments) having a relative difference of emphysema destruction above a threshold amount, while homogenous emphysema in a lung is characterized by any two or more regions (e.g., lobes, segments) having a relative difference of emphysema destruction below a threshold amount. In some patients, both right and left lungs may be heterogenous or homogenous, or one lung may be heterogenous while the other lung may be homogenous.

Pharmacological treatment can be prescribed for COPD. A treatment regimen of bronchodilators, B2-agonists, muscarinic agonists, corticosteroids, or combinations thereof may provide short term alleviation of the symptoms of COPD. Non-pharmaceutical management solutions, such as home oxygen, non-invasive positive pressure ventilation, and pulmonary rehabilitation, can also be used. Another treatment option for patients with severe emphysema is lung volume reduction surgery (LVRS). This surgery involves removing poorly functioning portions of a lung (typically up to 20 to 25 percent of lung volume) thereby reducing the overall size of the lung and making more volume within the chest cavity available for expansion of relatively healthy lung tissue. With greater available volume for expansion, the lung tissue remaining after LVRS has an enhanced capacity for effective gas exchange.

Procedures for lung volume reduction without surgical removal of diseased lung tissue also exist. Examples include use coils or clips to seize and physically compact diseased lung tissue. These procedures can reduce the overall volume of a lung for an effect similar to that of LVRS. Another device-based treatment for COPD involves placement of one-directional stent valves in airways proximal to emphysematous tissue. These valves allow air to flow out of but not into overinflated portions of the lung. Although not conventionally used to treat COPD, stents are sometimes used in the lumen of the central airways (e.g., the trachea, main bronchi, lobar bronchi, and/or segmental bronchi) to temporarily improve patency of these airways. For example, stents may be used to temporarily improve patency in a central airway affected by a benign or malignant obstruction.

Steam/vapor therapy, such as bronchoscopic thermal vapor ablation (BTVA), is yet another COPD treatment option. BTVA involves introducing heated water vapor into diseased lung tissue. This produces a thermal reaction leading to an initial localized inflammatory response followed by permanent fibrosis and atelectasis. Similar to thermal treatments like BTVA, there are also biochemical treatments that involve injecting glues or sealants into diseased lung tissue. Both thermal and biochemical procedures may precipitate remodeling that results in reduction of tissue and air volume at targeted regions of hyperinflated lung.

Some other known COPD treatments involve bypassing an obstructed airway. For example, a perforation through the chest wall into the outer portions of the lung can be used to create a direct communication (e.g., a bypass tract) between diseased alveoli and the outside of the body. If no other steps are taken, these bypass tracts will typically close by normal healing or by the formation of granulation tissue. Accordingly, placing a tubular prosthetic in the bypass tract can temporarily extend the therapeutic benefit.

### III. Endobronchial Implants

In some embodiments, the present technology provides endobronchial placement of an implant to establish or improve airway patency (also referred to herein as “endobronchial implant therapy”). The implant can incorporate features designed to minimize or reduce foreign body reaction, such as minimal surface area coverage (or at least significantly reduced surface area coverage compared to other types of implants), an open helical structure, and/or high contrast between outward radial force and flexibility along the longitudinal axis. Such endobronchial implants may also be referred to herein as minimal airway reinforcement implants or minimal endobronchial reinforcement implants. A minimal endobronchial reinforcement implant can be advantageous compared to other types of endobronchial implants (e.g., valves, airway stents) whose therapeutic effect can be undermined due to foreign body reaction (e.g., granulation tissue reaction, mucous impaction, airway constriction).

The implant can be placed at a treatment location including a previously collapsed airway, such as a previously collapsed distal airway. Deployment of the implant can release air trapped in a hyperinflated portion of the lung and/or reduce or prevent subsequent trapping of air in this portion of the lung. In at least some cases, it is desirable for a treatment location at which an implant is deployed to include an airway of generation 4 or higher/deeper, such as (from distal to proximal) the respiratory bronchioles, terminal bronchioles, conducting bronchioles, bronchioles or sub-segmental bronchi and then run proximally to a more central, larger airway (e.g., 6th generation or more proximal/lower) such as (from distal to proximal) sub-segmental bronchi, segmental bronchi, lobar bronchi and main bronchi. A single implant may create a contiguous path distal to proximal to reliably create passage for the trapped air. In an alternative embodiment, multiple, discrete implants can be used instead of a single, longer implant. The multiple, discrete implants may be placed in bronchial airways that have collapsed or are at risk of collapse. The use of multiple, discrete implants in select locations in the bronchial tree may have the advantage of using less material, thereby reducing contact stresses and foreign body response and allow for greater flexibility and customization of therapy. For example, whereas a single implant embodiment may run from a higher generation airway distally to a lower generation airway proximally, a system of multiple, discrete implants may allow for placement of implants in multiple airways of the same generation.

The devices, systems and methods described herein may be administered to different bronchopulmonary segments in order to release trapped air from regions of the lung in the safest and most efficient manner possible. For example, treatment of the left lung may involve one or more of the following segments: Upper Lobe (Superior: apical-posterior, anterior; Lingular: superior, inferior); Lower Lobe: superior, antero-medial basal, lateral basal. Treatment of the right lung may involve one or more of the following segments: Upper Lobe: apical, anterior, posterior; Middle Lobe: medial, lateral; Lower Lobe: superior, anterior basal, lateral basal. The treatments described herein may involve placement of a single implant in a single lung (right or left), a single implant in each lung or multiple implants in each lung. Treatment within a particular lung may involve placing an implant in a specific lobe (e.g., upper lobe) and a specific segment within such lobe or it may involve placement of at least one implant in multiple lobes, segments within a lobe or sub-segments within a segment. Determination of which parts of the lung to treat can be made by the clinical operator (e.g., pulmonologist or surgeon) with the assistance of imaging (e.g., CT, ultrasound, radiography, or bronchoscopy) to assess the presence and pathology of disease and impact on pulmonary function and airflow dynamics.

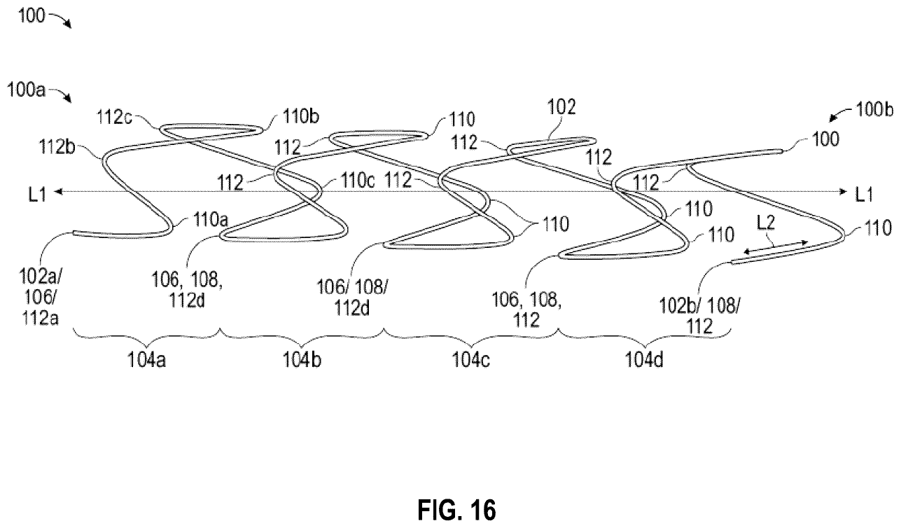

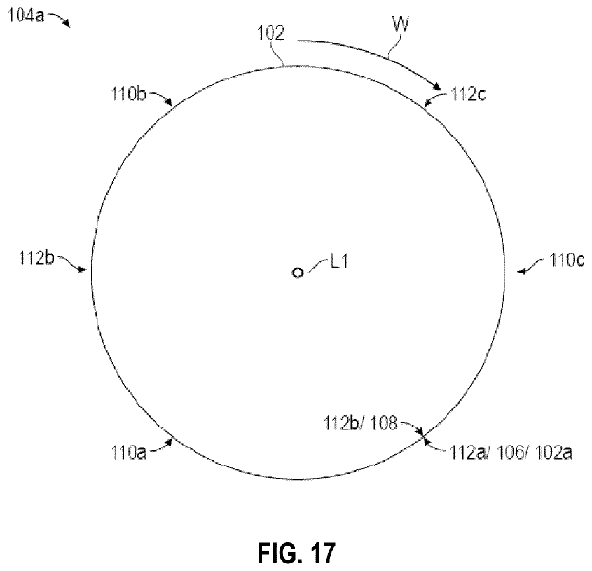

FIGS. 16 and 17 illustrate an example of a minimal endobronchial reinforcement implant configured as an expandable device 100 for placement in an airway lumen. In particular, FIG. 16 is a side view of the expandable device 100 in an expanded, unconstrained state, and FIG. 17 is an end view of the device 100. As shown in FIG. 16, the device 100 can comprise a generally tubular structure configured to be positioned within an airway lumen. For example, the device 100 may be configured to be implanted in an airway lumen such that the device 100 maintains a lumen of a minimum desired diameter in the airway. The device 100 has a first end portion 100a, a second end portion 100b opposite the first end portion 100a, and a central longitudinal axis L1 extending between the first and second end portions 100a, 100b. As used herein, the term “longitudinal” can refer to a direction along an axis that extends through the lumen of the device while in a tubular configuration, the term “circumferential” can refer to a direction along an axis that is orthogonal to the longitudinal axis and extends around the circumference of the device when in a tubular configuration, and the term “radial” can refer to a direction along an axis that is orthogonal to the longitudinal axis and extends toward or away from the longitudinal axis.

The device 100 can comprise an elongated member 102 wound about the longitudinal axis L1 of the device 100. In some embodiments, the elongated member 102 is heat set in a novel three-dimensional (3D) configuration such that the elongated member 102 is configured to self-expand to the preset configuration. In some embodiments, the elongated member 102 is not heat set and/or configured to self-expand. For example, the elongated member 102 is balloon-expandable. In some embodiments, the elongated member 102 is balloon-expandable and self-expanding. The elongated member 102 has a first end 102a and a second end 102b opposite the first end 102a along a longitudinal axis L2 of the elongated member 102. The elongated member 102 can comprise a wire, a coil, a tube, a filament, a single interwoven filament, a plurality of braided filaments, a laser-cut sheet, a laser-cut tube, a thin film formed via a deposition process, and other suitable elongated structures and/or methods, such as cold working, bending, EDM, chemical etching, water jet, etc. The elongated member 102 can be formed using materials such as nitinol, stainless steel, cobalt-chromium alloys (e.g., 35N LT®, MP35N (Fort Wayne Metals, Fort Wayne, Indiana)), Elgiloy, magnesium alloys, tungsten, tantalum, platinum, rhodium, palladium, gold, silver, or combinations thereof, or one or more polymers, or combinations of polymers and metals. In some embodiments, the elongated member 102 may include one or more drawn-filled tube (“DFT”) wires comprising an inner material surrounded by a different outer material. The inner material, for example, may be radiopaque material, and the outer material may be a superelastic material.

Although the device 100 shown in FIG. 16 comprises a single elongated member 102, the device 100 may comprise any number of elongated members 102. A single elongated member, such as a single wire expandable device, can be easier to remove and/or reposition as the operator can grab the elongated member on one end and pull it through a working channel of a scope. The elongated member will straighten out in either the balloon expandable or self-expanding form.

Referring to FIG. 16, the elongated member 102 may be wound about the longitudinal axis L1 of the device 100 in a series of windings or loops 104, four of which are shown in FIG. 16 and individually labeled 104a-104d. Each of the loops 104 can extend around the longitudinal axis L1 of the device 100 between a first end 106 and a second end 108. In some embodiments, the loops 104 are connected end to end such that, for example, a second end 108 of the first loop 104a is the first end 106 of the second loop 104b. The second end 108 can be disposed approximately 360 degrees from the first end 106 about the longitudinal axis L1 of the device 100. That is, the first and second ends 106, 108 can be disposed at generally equivalent circumferential positions relative to the longitudinal axis L1 of device 100. In some embodiments, the device 100 has a circular cross-sectional shape. In other embodiments, the device 100 may have other suitable cross-sectional shapes (e.g., oval, square, triangular, polygonal, irregular, etc.). The cross-sectional shape of the device 100 may be generally the same or vary along the length of the device 100 and/or from loop to loop.

The expanded cross-sectional dimension of the device 100 may be generally constant or vary along the length of the device 100 and/or from loop to loop. For example, as discussed herein, the device 100 can have varying cross-sectional dimensions along its length to accommodate different portions of the airway. For instance, the device 100 can have a first cross-sectional dimension along a first portion configured to be positioned in a more distal portion of the airway (such as, for example, in a terminal bronchiole and/or emphysematous areas of destroyed and/or collapsed airways), and a second cross-sectional dimension along a second portion configured to be positioned more proximally (such as in a primary bronchus and/or another portion that has not collapsed). The second portion, for example, can be configured to be positioned in a portion of the airway that is less emphysematous than the collapsed distal portion and/or has cartilage in the airway wall (preferably rings of cartilage and not plates), which can occur at the lobar (generation 2) or segmental (generation 3) level.

In some embodiments, the expanded cross-sectional dimension of the device 100 in an unconstrained (e.g., removed from the constraints of a catheter or airway), expanded state is oversized relative to the diameter of the native airway lumen. For example, the expanded, unconstrained cross-sectional dimension of the device 100 can be at least 1.5× the original (non-collapsed) diameter of the airway lumen in which it is intended to be positioned. In some embodiments, the device 100 has an expanded, cross-sectional dimension that is about 1.5× to 6×, 2× to 5×, or 2× to 3× the diameter of the original airway lumen. Without being bound by theory, it is believed that expanding the airway lumen to the greatest diameter possible without tearing the airway wall will provide the greatest improvement in pulmonary function (for example, as measured by outflow, FEV, and others).

As shown in FIG. 16, the elongated member 102 may undulate along its longitudinal axis L2 as it winds around the longitudinal axis L1 of the device 100, forming a plurality of alternating peaks 110 (closer to the second end portion 100b of the device 100) and valleys 112 (closer to the first end portion 100a of the device 100). At least some of the valleys 112 can be at different locations along the longitudinal axis L1 of the device 100 than at least some of the peaks 110. Additionally or alternatively, at least some of the valleys 112 can be at different longitudinal locations than at least some others of the valleys 112 and/or at least some of the peaks 110 can be at different longitudinal locations than at least some others of the peaks 110.

As an example, three peaks 110 and four valleys 112 of the first loop 104a have been individually labeled as peaks 110a-110c and valleys 112a-d. As shown in FIGS. 16 and 17, for the first loop 104a in the direction of the wind W, the elongated member 102 extends from the first end 106 of the elongated member 102, which comprises a first valley 112a of the first loop 104a, to a first peak 110a of the first loop 104a along a first longitudinal direction toward the second end portion 100b of the device 100. The elongated member 102 can then extend from the first peak 110a to a second valley 112b along a second longitudinal direction opposite of the first longitudinal direction, from the second valley 112b to a second peak 110b along the first longitudinal direction, from the second peak 110b to a third valley 112c along the second longitudinal direction, from the third valley 112c to a third peak 110c along the first longitudinal direction, and from the third peak 110c to a fourth valley 112d (which is also the second end 108 of the first loop 104a) along the second longitudinal direction. Thus, when traveling in a direction of the wind W around a given loop 104, the loop 104 does not consistently progress from the first end portion 100a of the device 100 to the second end portion 100b of the device 100 (or vice versa), but rather undulates so that along certain portions of its length, the loop 104 becomes progressively closer to the first end portion 100a of the device 100, and along other portions of its length the loop becomes progressively closer to the second end portion 100b of the device 100.

Although the first and second ends 106, 108 of one of the loops 104 may be generally aligned circumferentially, the first and second ends 106, 108 are longitudinally offset. The first peak 110a can be closer to the second end portion 100b of the device 100 than the first valley 112a. The second valley 112b can be closer to the first end portion 100a of the device 100 than the first peak 110a and/or the first valley 112a. The second peak 110b can be closer to the second end portion 100b of the device 100 than the second valley 112b, the first peak 110a, and/or the first valley 112a. The third valley 112c can be closer to the first end portion 100a of the device 100 than the second peak 110b and/or closer to the second end portion 100b of the device 100 than the first valley 112a and/or the second valley 112b. In some embodiments, the third valley 112c can be substantially longitudinally aligned with the first peak 110a. The third peak 110c can be closer to the second end portion 100b of the device 100 than the third valley 112c, the second peak 110b, the second valley 112b, the first peak 110a, and/or the first valley 112a. The fourth valley 112d can be closer to the first end portion 100a of the device 100 than the third peak 110c and/or closer to the second end portion 100b of the device 100 than the third valley 112c, the second valley 112b, the first peak 110a, and/or the first valley 112a. In some embodiments, the fourth valley 112d can be substantially longitudinally aligned with the second peak 110b.

Although FIGS. 16 and 17 show a device 100 comprising four loops 104, each having four peaks 110 and four valleys 112, in some embodiments one or more of the loops 104 has more or fewer peaks 110 and/or more or fewer valleys 112. For example, in some embodiments one or more of the loops 104 has one, two, three, four, five, six, seven, eight, etc. peaks 110 per loop 104 and one, two, three, four, five, six, seven, eight, etc. valleys 112 per loop 104. The loops 104 may have the same or a different number of peaks 110, and the loops 104 may have the same or a different number of valleys 112. A circumferential distance (e.g., an angular separation) between adjacent ones of the peaks 110 and valleys 112 can be uniform or non-uniform in a given loop 104. In some embodiments, adjacent ones of the peaks 110 and valleys 112 can be spaced apart around a circumference of the device 100 by about 90 degrees, about 120 degrees, about 150 degrees, about 180 degrees, about 210 degrees, about 240 degrees, about 270 degrees, about 300 degrees, and/or about 330 degrees. In addition, the amplitude of the peaks 110 may be the same or different along a given loop 104 and/or amongst the loops 104, and the amplitude of the valleys 112 may be the same or different along a given loop 104 and/or amongst the loops 104. Moreover, the peaks 110 and valleys 112 can have the same or different amplitudes.

As shown in FIG. 16, a portion of the elongated member 102 between adjacent peaks 110 and valleys 112 can be linear, curved, or both. Adjacent portions of the elongated member 102 between two sets of adjacent peaks 110 and valleys 112 can form a V-shaped and/or U-shaped structure. At least some of the valleys 112 can be concave toward the second end portion 100b of the device 100 and/or at least some of the peaks 110 can be concave toward the first end portion 100a of the device 100.

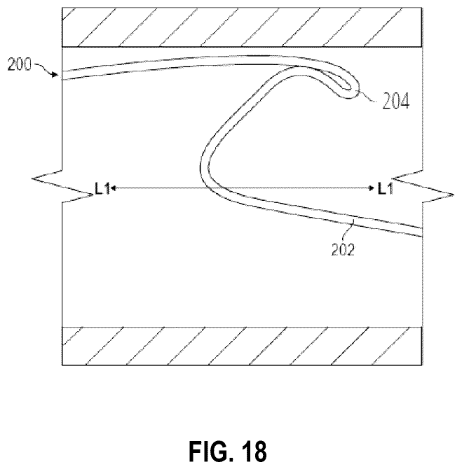

In some embodiments, for example as shown in FIG. 16, the elongated member 102 can extend around a circumference of the device 100 and/or along a longitudinal axis L1 of the device 100, without substantially extending radially away or towards the longitudinal axis L1. Still, in some embodiments, a device 200 can comprise an elongated member 202 that undulates radially with respect to a longitudinal axis L1 of the device 200. As shown in FIG. 18, for example, the elongated member 202 can form peaks 204 and/or valleys 204 that are located closer to the longitudinal axis L1 than intermediate portions of the elongated member 202 between the peaks 204 and valleys 204. The apices of each “V” can be bent radially inward toward the center of the lumen, so that only the longitudinally-extending portions of the elongated member 202 are touching the bronchial wall. Such a configuration can prevent the stent from impeding mucus flow along the wall of the bronchus.

The radial mechanism of expansion allows the expandable device 200 to be easily designed and delivered by both self-expansion and balloon-expansion. The zig-zag pattern of the devices disclosed herein, including the example shown in FIG. 16, is configured to conform to different diameter airways with a single design, whereas conventional coils are a fixed diameter. This is especially advantageous for achieving gradual airway dilation over time. The expandable device stores expansion potential in the implant design which is achieved via beams that bend and elastic potential is established. The expandable device in balloon expandable form also has a unique potential to form a coil by expanding the zig-zags all the way to a straight line when geometrically designed this way.

In some embodiments, the devices described herein include at least one material configured to facilitate visualization, such as a radiopaque material. The material can be the same material used to form the device, or can be incorporated into the device via doping, coating, attachment of a separate component incorporating the material (e.g., a radiopaque marker), etc.

Additional examples of devices suitable for use with the present technology are described in International Application No. PCT/US2022/073962, the disclosure of which is incorporated herein by reference in its entirety.

### IV. Methods for Patient Selection, Treatment Planning, and Monitoring

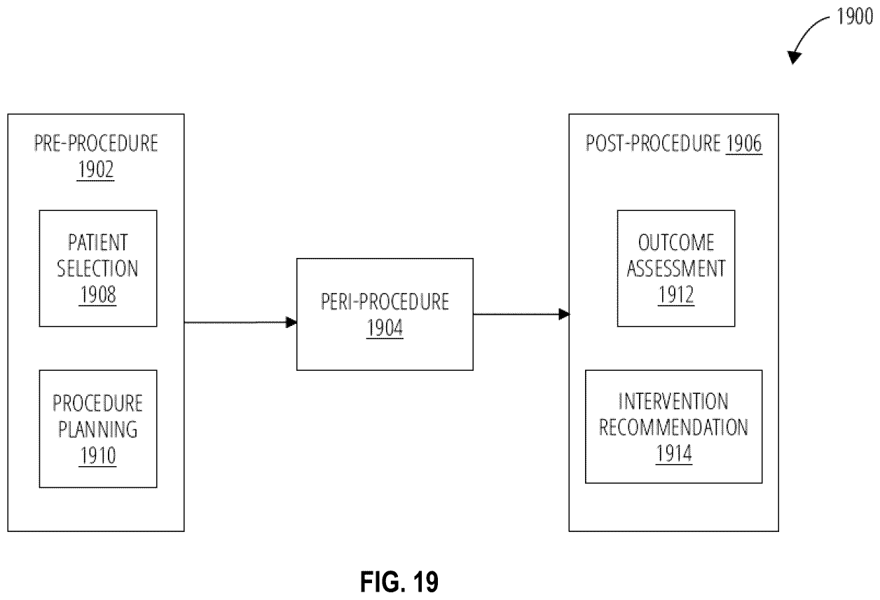

FIG. 19 is a block diagram providing a general overview of a workflow 1900 for the selection of patients for treatment as well as planning and monitoring a treatment procedure, in accordance with embodiments of the present technology. For example, the treatment procedure can be a procedure for treating a patient having a pulmonary disease (e.g., COPD) by placing one or more implanted devices into one or both lungs of the patient. The devices can be any of the embodiments of endobronchial implants described herein, such as a minimal endobronchial reinforcement implant.

As shown in FIG. 19, the workflow 1900 can be divided into a pre-procedure phase 1902, a peri-procedure phase 1904, and a post-procedure phase 1906. In some embodiments, the pre-procedure phase 1902 occurs before the patient has been diagnosed with a pulmonary disease. Alternatively, the pre-procedure phase 1902 can occur after the patient has been diagnosed with the pulmonary disease, but before the patient has received treatment (e.g., endobronchial implant therapy and/or other therapy) for the pulmonary disease.

The pre-procedure phase 1902 can involve determining whether the patient is a candidate for a treatment for the pulmonary disease (block 1908). For example, the treatment can be or include an airway treatment for COPD. The airway treatment can include pharmacological treatment (e.g., bronchodilators), interventional treatment (e.g., implant and/or non-implant procedures), or combinations thereof. In some embodiments, the interventional treatment includes vapor therapy (e.g., BTVA), administration of a sealant, transbronchial fenestration (e.g., airway bypass stents), and/or endobronchial implant therapy (e.g., placement of an endobronchial coil, placement of an endobronchial valve, and/or placement of a minimal endobronchial reinforcement implant).

The process of block 1908, also referred to herein as “patient selection,” can involve analyzing patient data (e.g., CT data and/or other image data, medical records, questionnaires, other diagnostics) to assess the current state of the patient. For instance, the patient selection process can determine whether the patient has or is at risk of developing a pulmonary disease, and, optionally, the disease profile (e.g., type, locations, severity). Optionally, patient data obtained over time can be used to track disease progression. The patient selection process can also involve evaluating whether the patient is a good candidate for a particular treatment for the pulmonary disease, e.g., the predicted likelihood of achieving a successful treatment outcome with the treatment.

In some embodiments, the patient selection process involves analyzing patient data and predicting the degree of response, based on factors such as homogeneity/heterogeneity of emphysema and/or collateral ventilation, locations of diseased portions (e.g., tissue destruction, air trapping), proximity to anatomical structures (e.g., pleura, heart, nodules), proximity to other medical devices, appropriate bronchial pathways to target(s), alveolar collapse, diaphragm movement, where air flows in response to breath and/or flow patterns, and/or calculated measures of lung volume and/or health (e.g., RV, TLC, RV/TLC ratio). Optionally, the patient selection process can involve screening the patient data for exclusionary characteristics, such as tumors, lesions, giant bullae, central airway collapse, etc.

In some embodiments, the patient selection process involves predicting patient outcomes by comparing the patient's disease profile against aggregated, normalized data of other patients. This approach can be used to predict how the patient's quality of life may decline over time (e.g., creating trendlines), and/or how the patient may respond to different treatments (e.g., pharmacological; interventional such as valves, coils, steam, hydrogel glue, thermal ablation, non-thermal ablation; surgical) in comparison with endobronchial implant therapy using a minimal endobronchial reinforcement implant.

In some embodiments, the patient selection process of block 1908 is implemented at least in part by a patient engagement utility. The patient engagement utility can be a software module that administers an automated questionnaire relating to symptoms and/or quality of life metrics. Based on the patient's answers, the patient engagement utility can put in orders for imaging (e.g., CT, X-Ray, magnetic resonance imaging (MRI), single-photon emission computerized tomography (SPECT), bronchoscopy) and/or tests (spirometry, arterial blood gas). Based on the image data and/or test results, the patient engagement utility can generate an interactive patient report that provides a COPD “risk” assessment (e.g., X % likelihood that the patient has COPD) and a referral to a respiratory physician. The patient report can also include information on available treatments and predictions of potential benefit of the treatments, based on the patient's provisional disease profile. For instance, the patient report can provide personalized, evidence-based recommendations that are understandable by laypersons (e.g., if the patient gets treatment, their quality of life can be restored by X %, they will be able to walk two flights of stairs versus one flight previously), which can promote patient driven market development.

Optionally, the patient engagement utility can send the referral to the respiratory physician, along with a detailed physician report with the data, analytics, and risk assessment generated for the patient. The physician report can include a preliminary diagnosis, recommendations for additional and/or confirmatory testing, and/or predictive analytics related to progression of disease, responsiveness to medical and/or pharmacological treatment, interventional treatment, surgery, etc. Optionally, the physician report can also include a referral to an interventionalist, if appropriate.

If the patient is determined to be a good candidate for treatment, the pre-procedure phase 1902 can generate a plan for a treatment procedure for the patient (block 1910). The process of block 1910, also referred to herein as “procedure planning,” can involve determining parameters of a treatment plan. For instance, parameters for endobronchial implant therapy (e.g., using a minimal endobronchial reinforcement implant) can include the number of implants, implant size (e.g., length, outer diameter), implant geometry, other implant characteristics (e.g., flexibility, material, design features), placement location (e.g., which airway(s), segment(s), sub-segment(s), lobe(s), etc. to place the implant in), placement pathway, and/or other treatment solutions that can be used in combination with endobronchial implant therapy (e.g., medications, surgery, other medical devices). The selection of the implant configuration can be based on factors such as the outer diameter of the target airway, wall thickness, lumen inner diameter, proximity to anatomical structures and/or the airway generation. The treatment plan can also include information such as the airway(s) to place the implant in, how the implants are predicted to supply air to areas of the lung, airway wall structure and sizing, how airway wall structure and sizing is predicted to interface with the implant, proximity to structures, airway mechanical strength (e.g., radius, wall thickness), and/or types of tissues (e.g., fat, muscle, connective tissue-which may suggest implant characteristics). In some embodiments, the procedure planning process is part of a software tool used by an interventionalist.

In some embodiments, the procedure planning process involves modeling the patient's lungs, e.g., before and after implant placement. For instance, a 3D model of the bronchial tree can be optionally generated from CT data and/or other image data. In some embodiments, CT data can identify the most diseased lobes (and/or segments, sub-segments, etc.) and help with identifying incomplete fissures, but may be limited in identifying the exact airways that are most impactful due to resolution, and thus may be combined with higher resolution imaging modalities such as MRI to allow for assessment of specific airways. Moreover, it may not be necessary to determine a precise airway target since any opened airway that creates a connected expressway from peripheral to a proximal airway having integrity may be sufficient to release trapped air, particularly if collateral ventilation further facilitates air movement through parenchyma.

The 3D model can be used to model various treatment options and the associated results (e.g., placement of a valve at a target location produces an X % increase in FEV₁, whereas placement of a minimal endobronchial reinforcement implant produces a Y % increase in FEV₁). The modeling results can be used to select the appropriate therapy type, target location(s) for implant placement (e.g., target airways, segments, and/or lobe), and/or sequence of target locations. Modeling techniques (e.g., computational flow dynamics, AI-based approaches) can also be used to identify optimal target airways and/or predict an optimal implant plan based on factors such as predicted effectiveness for removing trapped air, regions with trapped air, presence or proximity to incomplete fissures, safety for avoiding iatrogenic injury (e.g., damage to pleura, blood vessel, organs), safety and durability for reducing implant fatigue, safety and effectiveness for reducing risk of friction against adjacent implants, minimizing the number of implants (e.g., for purposes of safety, ease of use, reducing procedure time), minimizing the amount of foreign material (e.g., for safety purposes), minimizing the number of bends, minimizing the risk of implant rejection or migration (e.g., due to coughing), and/or minimizing procedure time. Modeling can also be used to predict how the lung may behave with endobronchial implant therapy. For example, modeling can be used to predict which lung segments will have reduced air trapping and, accordingly, contribute less to residual volume compared to the baseline (e.g., pre-treatment) measurements, and/or which lung segments may contribute more to improved pulmonary function. It is anticipated that lung segments that have less disease and are proximate or adjacent to lung segments with substantial air trapping may be able to expand substantially compared to baseline and, accordingly, may contribute most to improved pulmonary function. Additionally, modeling may be useful in predicting risk of pneumothorax. Analysis of inspiratory and expiratory CT may be able to detect the presence and location of adhesions which are at risk of tearing from the pleural wall following treatment. This perforation and pneumothorax risk analysis associated with predictive modeling may inform the location and sequence of treatment.

The peri-procedure phase 1904 can occur during the treatment procedure, immediately before the procedure (e.g., when preparing for the procedure), and/or immediately after the procedure (e.g., when assessing the immediate outcomes of the procedure). The peri-procedure phase 1904 can involve using data produced during the pre-procedure phase 1902 to assist the interventionalist in performing the treatment procedure. For instance, 3D models of the patient anatomy generated during the pre-procedure phase 1902 can be integrated with peri-procedural visualization (e.g., fluoroscopy, bronchoscopy) and navigation technology (e.g., robotic navigation and delivery systems) to enhance delivery and targeting of an endobronchial implant to the appropriate location in the lung.

In some embodiments, the peri-procedure phase 1904 involves modifying and/or augmenting the treatment plan during the procedure by assessing the outcome of a previous step in the plan and providing recommendations for next steps. For instance, real-time data characterizing the patient's response to a previously placed implant can be used to demonstrate procedure success, evaluate whether the procedure should continue as planned, and/or determine whether modifications should be made (e.g., repositioning of the implant, removal of the implant, placement of additional implants, administration of other therapies). The real-time data can include, for example, image data, AI analyses, physician input, pressure and/or flow measurements, physiological metrics (e.g., O₂ saturation, breathing metrics), etc. Optionally, the real-time data can be generated by a ventilator or other device that examines the correlation between data such as O₂ saturation and air flow, and uses such data as a metric of success. This approach can improve efficacy and reduce procedure time.

The post-procedure phase 1906 can occur after the treatment procedure, such as at least 24 hours, 48 hours, 1 week, 2 week, 3 weeks, 4 weeks, 1 month, 2 months, 3 months, 6 months, or 1 year after the procedure. In some embodiments, the post-procedure phase 1906 involves assessing a treatment outcome of the patient (block 1912) at one or more timepoints after the procedure (e.g., at a single timepoint following treatment, or repeatedly following treatment, such as periodically or intermittently). The process of block 1912, also referred to herein as “outcome assessment,” can involve analyzing patient data (e.g., CT data and/or other image data, medical records, questionnaires, other diagnostics) to evaluate the patient's response to treatment, such as whether the patient's condition is improving, stable, or deteriorating over time. For instance, post-procedure lung function metrics can be compared to pre-procedure lung function metrics, such as airway patency, lung/lobe/segment volume in inspiration and expiration, RV, TLC, RV/TLC ratio, flow through targeted airways, and/or flow through adjacent airways. Optionally, the state of the patient's lung can be assessed by determining a set of lung metrics from the patient data (e.g., CT data of the lung), then comparing the set of lung metrics to a set of second lung metrics determined from other types of data, such as image data of the same lung before treatment (e.g., a baseline CT image obtained before placement of an endobronchial implant), image data of the same lung at an earlier time point after treatment (e.g., comparison of 6 month follow up and 12 month follow up CT images after placement of an endobronchial implant), image data of the same lung after placement of another endobronchial implant at a different location than the location of the current endobronchial implant, and/or a library of lung image data of patients having COPD. The outcome assessment process can also involve evaluating the state of the endobronchial implant over time, such as whether the implant is still positioned at the target location and functioning as intended, whether the implant has migrated, whether the implant has collapsed or otherwise failed, etc.

In some embodiments, the process of block 1912 can include transforming some or all of the patient data into a “virtual bronchoscopy” with which a user may interact. For example, a 3D model of the lung (including airways and placed implant(s)) can be reconstructed from imaging (e.g., follow up CT imaging) of the lung post-procedure. In some embodiments, for example, developing a virtual bronchoscopy can include obtaining CT slice images of at least a portion of a lung in which an endobronchial implant is placed, reconstructing a 3D model of the lung based on the CT slice images, and segmenting the 3D model to differentiate various structures in the lung including cardiopulmonary structures (e.g., 3D airway structures and/or pulmonary vasculature) and the placed endobronchial implant. Additionally or alternatively, the CT slice images can be segmented to differentiate between structures in the lung prior to 3D model construction. Such segmentation can, for example, be based at least in part one density differences between different cardiopulmonary features and the endobronchial implant itself, as represented as different voxel densities in the CT images.

The 3D model can be displayed on a suitable display (e.g., on a computing device) and/or navigable by a user in a virtual bronchoscopy interaction. For example, the 3D model can be displayed on a monitor and/or on a wearable device (e.g., glasses, headset, goggles, etc.). In some embodiments, the 3D model can additionally or alternatively be displayed in an augmented reality (AR) and/or virtual reality (VR) environment. The 3D model can be navigated with a suitable user interface device (e.g., mouse, joystick, handheld controllers, buttons, scroll wheel or scroll balls, etc.).

In some embodiments, the display of the 3D model may include a highlighting or other emphasis of one or more implant features. For example, the implant may be visually indicated with an outline of the implant itself (e.g., colored line, or thicker line weight). As another example, one or more individual implant features may additionally or alternatively be visually indicated with markers associated with relevant implant features (e.g., markers corresponding to the proximal and distal ends of an endobronchial implant, outline of cross-sectional profile of an endobronchial implant at one or more locations along the airway in which the endobronchial implant is placed). In some embodiments, the implant outline and/or markers associated with individual implant features may be toggled on and/or off for display, such as to enable clearer visualization of certain features in the 3D model.

A virtual bronchoscopy can provide more detailed information regarding the lung, airways, and/or implant than what otherwise can be visually observed during a non-virtual bronchoscopy, or existing virtual bronchoscopy technologies. For example, in many instances, the placed implant may be configured to blend into the contours of the airways and minimize the induction of foreign body reactions, so the implant may not be easily visible on the tissue surface during a non-virtual bronchoscopy. In contrast, a virtual bronchoscopy in accordance with the present technology that allows navigation of a reconstructed 3D model of the lung, airways, and/or placed implant, as described above, can allow visualization and investigation of the airways, the placed implant, implant-airway tissue interactions, and the lung as a whole, even beyond the airway surface. In particular, visualization of the placed implant relative to its surroundings may be helpful for assessing treatment outcome for the patient post-procedure. In some embodiments, assessments of treatment outcome using the virtual bronchoscopy can be performed manually (e.g., by a user operating and navigating the virtual bronchoscopy), and/or with a software algorithm such as a trained machine learning algorithm (e.g., similar to those described herein).

In some embodiments, the outcome assessment process of block 1912 involves predicting future outcomes of the patient, such as the predicted disease progression, therapeutic benefits, implant state, etc. For instance, the outcome assessment process can predict whether any post-procedural issues are likely to arise, such as physiological issues (e.g., excessive mucus, granulation tissue, and/or fibrosis) as well as issues with the implant (e.g., collapse, displacement, and/or other failure). If any issues are predicted to arise, the post-procedure phase 1906 can generate recommendations for interventions to prevent, mitigate, or otherwise address such issues (block 1914). The process of block 1914, also referred to herein as “intervention recommendation,” can produce recommendations for additional treatment procedures such as cleanup bronchoscopy, implant removal, implant replacement, placement of additional implants, consultations with healthcare professionals, etc.

In some embodiments, patient data is collected during the post-procedure phase 1906 using at-home devices, such as take-home spirometers and/or wearable devices (e.g., smart watches with sensors for blood oxygen levels, heart rate, activity (such as steps), altitude, position, and/or sleep; wearable stethoscopes that analyze lung sounds to detect early signs of disease exacerbation)). The data generated from such devices can be used to track the patient over extended time periods (e.g., weeks, months) and can allow for remote monitoring, thus reducing the frequency of doctor visits. Optionally, if the data indicates that there are potential health concerns (e.g., the patient's condition suddenly deteriorates), the patient can be instructed to see the doctor for follow-up.

Any of the processes of FIG. 19 (e.g., the patient selection process, procedure planning process, outcome assessment process, and/or intervention recommendation process) can be performed using one or more software algorithms, such as rule-based algorithms, machine learning algorithms, or combinations thereof. Examples of machine learning algorithms that may be used include: regression algorithms (e.g., ordinary least squares regression, linear regression, logistic regression, stepwise regression, multivariate adaptive regression splines, locally estimated scatterplot smoothing), instance-based algorithms (e.g., k-nearest neighbor, learning vector quantization, self-organizing map, locally weighted learning), regularization algorithms (e.g., ridge regression, least absolute shrinkage and selection operator, elastic net, least-angle regression), decision tree algorithms (e.g., Iterative Dichotomiser 3 (ID3), C4.5, C5.0, classification and regression trees, chi-squared automatic interaction detection, decision stump, M5), Bayesian algorithms (e.g., naïve Bayes, Gaussian naïve Bayes, multinomial naïve Bayes, averaged one-dependence estimators, Bayesian belief networks, Bayesian networks, hidden Markov models, conditional random fields), clustering algorithms (e.g., k-means, single-linkage clustering, k-medians, expectation maximization, hierarchical clustering, fuzzy clustering, density-based spatial clustering of applications with noise (DBSCAN), ordering points to identify cluster structure (OPTICS), non negative matrix factorization (NMF), latent Dirichlet allocation (LDA), Gaussian mixture model (GMM)), association rule learning algorithms (e.g., apriori algorithm, equivalent class transformation (Eclat) algorithm, frequent pattern (FP) growth), artificial neural network algorithms (e.g., perceptrons, neural networks, back-propagation, Hopfield networks, autoencoders, Boltzmann machines, restricted Boltzmann machines, spiking neural nets, radial basis function networks), deep learning algorithms (e.g., deep Boltzmann machines, deep belief networks, convolutional neural networks, stacked auto-encoders), dimensionality reduction algorithms (e.g., principle component analysis (PCA), independent component analysis (ICA), principle component regression (PCR), partial least squares regression (PLSR), Sammon mapping, multidimensional scaling, projection pursuit, linear discriminant analysis, mixture discriminant analysis, quadratic discriminant analysis, flexible discriminant analysis), ensemble algorithms (e.g., boosting, bootstrapped aggregation, AdaBoost, blending, gradient boosting machines, gradient boosted regression trees, random forest), or suitable combinations thereof. The machine learning algorithms described herein can be trained using any suitable technique, including supervised learning, unsupervised learning, semi-supervised learning, reinforcement learning, or suitable combinations thereof.

In some embodiments, the software algorithms described herein include at least one computer vision algorithm, which may or may not be a machine learning algorithm (e.g., a deep learning algorithm such as a convolutional neural network). The computer vision algorithm can receive image data as input, and can generate output data characterizing one or more objects present in the image data. For instance, the computer vision algorithm can receive CT data of one or both of the patient's lungs, and can identify objects in the lung such as anatomical structures, healthy tissues, diseased tissues, implanted devices (e.g., endobronchial implants), etc.

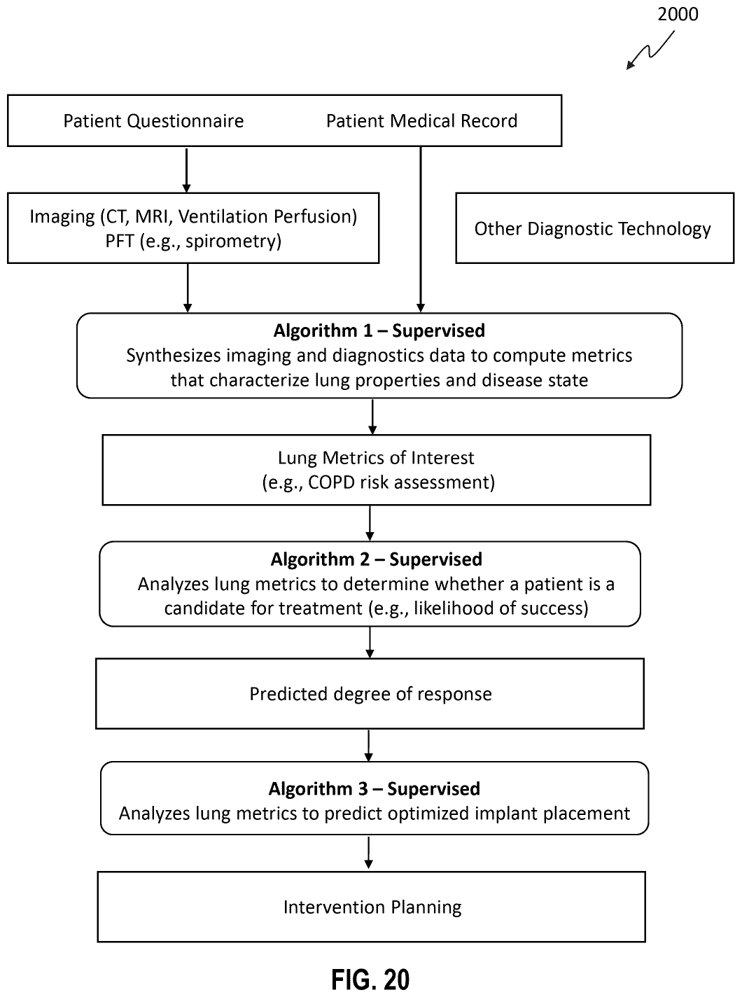

FIG. 20 is a flow diagram illustrating a method 2000 for planning a treatment for a patient, in accordance with embodiments of the present technology. The method 2000 can be performed as part of the pre-procedure phase 1902 of the workflow 1900 of FIG. 19. The method 2000 can involve receiving patient data, such as questionnaire information, medical record information, image data, pulmonary function test (PFT) data, and/or data from other diagnostic technologies. The method 2000 can implement one or more software algorithms that use the patient data to determine whether patients are candidates for endobronchial implant therapy (and/or other therapies for pulmonary diseases) and, optionally, assist with planning such therapies.

The questionnaire information can include the patient's responses to one or more questionnaires, which may be administered by a patient engagement utility as described above. The medical record information can include information from electronic health records for the patient, such as the patient's name, date of birth, demographic information, height, weight, medical history, familial medication history, symptoms, comorbidities, diagnoses, medications, test results, previous treatments and outcomes, and so on. The questionnaire information and/or medical record information can provide any of the following information: whether the patient has a cough, whether the patient is a smoker, whether the patient finds it hard to breathe, whether the patient is able to take deep breaths, whether the patient is able to perform typical activities (e.g., walking, showering), whether the patient has exacerbations, whether the patient has significant mucus, whether patient has had lung infections, and/or the patient's current and/or past drug regimen.

The image data can include data generated by any suitable imaging modality, such as CT, X-ray (e.g., chest radiography, fluoroscopy), MRI (e.g., ³He MRI, ¹²⁹Xe MRI), SPECT, bronchoscopy, ultrasound, ventilation-perfusion scan data, photographs (e.g., from multiple external cameras to map the surface topography of the chest), etc. For example, the CT data can include inspiratory CT data (e.g., obtained at the end of full inspiration) and/or expiratory CT data (e.g., obtained at the end of forced expiration). In some embodiments, the image data can be generated after the administration of a contrast agent in the patient (e.g., via intrapleural injection, inhalation, etc.), where the contrast agent can help provide contrast enhancement in CT imaging. For example, for pulmonary CT, suitable contrast agents include iodinated compounds (e.g., derivatives of diatrizoic acid), barium, radiolabeled albumin to track blood flow in the lungs, and labeled gases (e.g., Xenon-133) for tracking ventilation in the lungs. The CT data can include a series of 2D cross-sectional images of the patient's anatomy, with each image having a specified slice thickness and spaced apart by a specific slice interval (e.g., 10 mm intervals). For example, the 2D cross-sectional images can have a slice thickness of at least 3 mm (e.g., within a range from 3 mm to 10 mm), or can have a slice thickness of less than 3 mm, such as within a range from 1 mm to 2 mm (e.g., 1 mm). In some embodiments, the 2D cross-sectional images can be combined to generate a 3D volumetric CT model of the imaged lung tissue.

The PFT data can include any data characterizing the function of the patient's lungs, such as spirometry data, plethysmography data, exercise test results (e.g., 6 minute walk distance test), etc. The PFT data can include measurements of any of the following: tidal volume, minute volume, VC, functional residual capacity, residual volume (RV), total lung capacity (TLC), RV/TLC ratio, forced vital capacity (FVC), forced expiratory volume (FEV) (e.g., FEV in 1 second (FEV₁)), forced expiratory flow, peak expiratory flow, closing volume (CV), inspiratory capacity (IC), IC/TLC ratio, and/or diffusion capacity for carbon monoxide.

The other data from other diagnostic technologies can include data from one or more sensors configured to monitor the patient's state, which can include implanted sensors, non-invasive sensors, wearable sensors, or suitable combinations thereof. Examples of sensors suitable for use with the present technology include, but are not limited to, impedance sensors, pressure sensors, flow sensors, and wearable stethoscopes. As another example, bronchoscopy can be used for automated collection of information at specific locations in the lungs, such as information regarding flow, pressure, granulation tissue, fibrosis, mucus, epithelialization, and/or obstruction.

Any of the patient data types described herein can be obtained over a plurality of time points, such as two, three, four, five, 10, 20, 50, or more time points. For instance, patient data can be obtained over a plurality of time points spanning multiple seconds, minutes, hours, days, weeks, months, and/or years. In some embodiments, patient data is obtained at two or more of the following time points: before treatment (e.g., before placement of an endobronchial implant, before administration of a bronchodilator), after treatment (e.g., after placement of an endobronchial implant, after administration of a bronchodilator), before exercise, during exercise, after exercise, and/or during different phases of the respiratory cycle (e.g., inspiration, expiration). In other embodiments, however, some or all of the patient data can be obtained at a single time point.

As shown in FIG. 20, the patient data can be provided to a first software algorithm. The first software algorithm can be a first machine learning algorithm that has been trained (e.g., via supervised learning) to synthesize the patient data to compute metrics that characterize lung properties and/or the patient's disease state. In some embodiments, the first software algorithm can additionally or alternatively include other suitable automated processes. The output of the first software algorithm can be a set of lung metrics representing a state of one or both lungs of the patient. For example, the lung metrics can characterize any of the following lung parameters: FEV (e.g., FEV₁), FVC, VC, IC, IC/TLC ratio, functional residual capacity, TLC, diffusion capacity for carbon monoxide, RV, RV/TLC ratio, CV, lobar and/or segmental tissue destruction, lobar and/or segmental air trapping, lobar and/or segmental fissure status, extent of lobar and/or segmental fissure completion, lobar and/or segmental ventilation, lung function (e.g., regional assessment), disease phenotype (e.g., homogeneity/heterogeneity of lobar and/or segmental emphysema, type of emphysema (such as centriacinar emphysema, panacinar emphysema, or paraseptal emphysema), locations of diseased portions of the lung), lobar volume, segmental volume, segment locations, diaphragm shape, tissue density, opacity, proximity of diseased portions to anatomical structures (e.g., pleura, heart, nodules), proximity of diseased portions to other medical devices, lumen inner diameter of bronchial sections, air flow mapping, collapsed airways, airway pressure, airway compliance, pleural pressure, airway diameter to wall thickness, airway wall deformation, vascular perfusion, parenchyma density, bullae, information that localizes the disease in the lung (e.g., at the segmental and/or subsegmental levels), obstruction score mucus score, and/or degree of epithelialization. The lung metrics can characterize any of the above lung parameters at a single time point, and/or can characterize a change in any of the above lung parameters over a plurality of time points (e.g., before endobronchial implant therapy, after endobronchial implant therapy, before administration of a bronchodilator, after administration of a bronchodilator, before exercise, or during exercise). The lung metrics can correlate to whether the patient has a pulmonary disease such as COPD. Optionally, the lung metrics can be used to generate a risk assessment for the pulmonary disease (e.g., a percent likelihood that the patient has COPD).

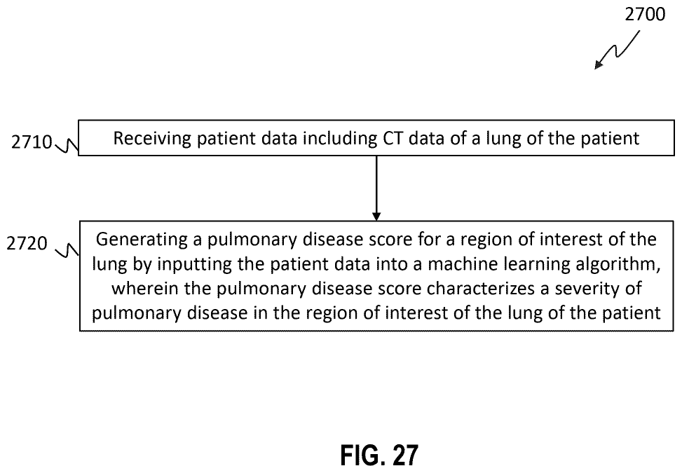

Additionally or alternatively, the lung metrics can be used to generate a disease “score” characterizing severity of pulmonary disease and/or representing a predictor of patient response to a treatment of the pulmonary disease. For example, the disease score can represent degree of emphysema destruction, extent of hyperinflation, and/or extent of air trapping in one or more regions of the lung. In some embodiments, the lung metrics can include one or more disease scores, where each disease score corresponds to a respective region of the lung (e.g., a particular lung, a particular lobe, a particular segment, a particular sub-segment). In some embodiments, the lung metrics can include a single disease score at least partially based on multiple “local” disease scores each corresponding to a respective region of the lung (e.g., a particular lung, a particular lobe, a particular segment, a particular sub-segment). For example, a single disease score can be an average value of the multiple local disease scores, or be based on any suitable calculation incorporating the multiple local disease scores. FIG. 27 is a flow diagram illustrating a method 2700 for evaluating a patient using such a disease score lung metric, including receiving patient data including CT data of a lung of the patient (block 2710), and generating a pulmonary disease score for a region of interest of the lung by inputting the patient data into a machine learning algorithm (block 2720).

In some embodiments, as described above, lung metrics produced by the first software algorithm can include disease phenotype. For example, the distribution of disease scores can be used to characterize homogeneity or heterogeneity of emphysema. In some embodiments, for example, a difference of disease score in ipsilateral lung regions (e.g., in different lobes, segments, and/or sub-segments in a particular lung) below a threshold difference value can correspond to a characterization of homogenous emphysema for that lung, while a difference of disease score in ipsilateral lung regions (e.g., in different lobes, segments, and/or sub-segments in a particular lung) above a threshold difference value can correspond to a characterization of heterogenous emphysema for that lung. In some embodiments, the threshold difference value used to characterize emphysema homogeneity/heterogeneity can range from about 10% to about 25%, from about 10% to about 20%, or from about 10% to about 15%. Additionally or alternatively, in some embodiments, the distribution of disease scores across a lung or lung region (e.g., across different lobes and/or different segments and/or different sub-segments) can be used to determine emphysema type. In particular, such lung metrics regarding emphysema tissue destruction at the segmental level and/or sub-segmental level may advantageously provide more insight into the disease and lung anatomy of a patient on a more granular level than analysis solely performed at the lobar level.

Additionally or alternatively, in some embodiments as described above, lung metrics produced by the first software algorithm can include characterization of lobar and/or segmental fissure status and/or completion, and/or lobar and/or segmental collateral ventilation. In particular, such lung metrics regarding fissure status, fissure completion, and/or ventilation at the segmental level may advantageously provide more insight into the disease and lung anatomy of a patient on a more granular level than analysis solely performed at the lobar level.

In some embodiments, lung metrics produced by the first software algorithm can be based at least in part on patterns of voxel density from one or more 3D models of patient tissue generated from inspiratory and/or expiratory CT scans. In general, voxel density is proportional to the attenuation of an X-ray beam through the tissue, which generally corresponds to the physical density of the tissue and other substances (e.g., air) being imaged. Voxel density can be represented in Hounsfield units (HU), which indicate along the Hounsfield scale the amount of X-ray attenuation that occurs in tissue corresponding to a particular voxel. In some embodiments, the first software algorithm can include evaluating voxel density across one or more lung regions relative to one or more voxel density thresholds, to characterize emphysema occurring on a lobar and/or segmental level, air trapping on a lobar and/or segmental level, the lobar and/or segmental lung volumes, amount of perfusion on a lobar and/or segmental level, and/or airway dimensions (e.g., lumen inner diameter of segmental airways along the length of airway pathways, airway wall thickness, etc.). The first software algorithm can incorporate different voxel density thresholds associated with different types of CT scans and/or different lung metrics. In some embodiments, the first software algorithm can output lung metrics based on analysis of both inspiratory CT scans and expiratory CT scans. In some embodiments, the first software algorithm can output lung metrics based on an expiratory CT scan, but not an inspiratory CT scan. In some embodiments, the first software algorithm can output lung metrics based on an inspiratory CT scan, but not an expiratory CT scan. Additionally or alternatively, the first software algorithm can include other suitable quantitative CT (QCT) techniques, such as those described in further detail herein.

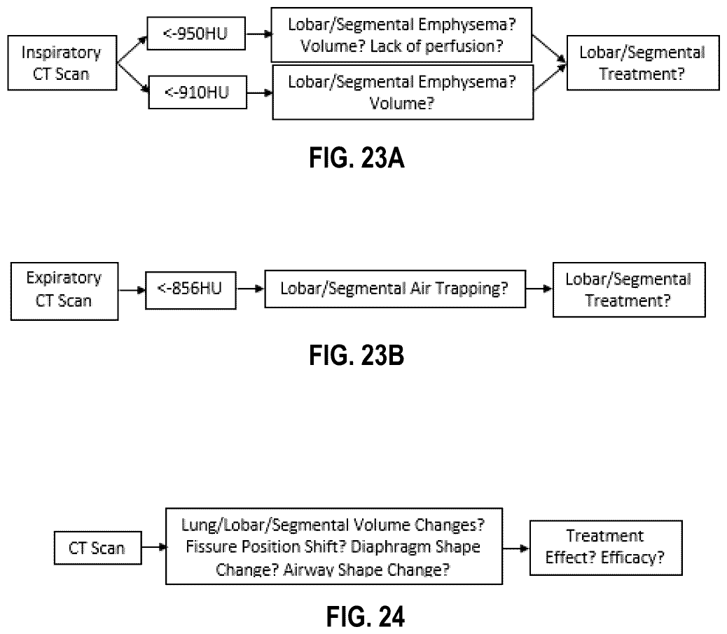

For example, as shown in FIG. 23A, the first software algorithm can incorporate one or more voxel density thresholds associated with an inspiratory CT scan. In some embodiments in which CT images from an inspiratory CT scan have a slice thickness of about 3 mm or greater (e.g., between 3 mm and 10 mm), emphysema can be quantified or otherwise characterized by assessing lung voxels on an inspiratory CT scan having attenuation below −910 HU (e.g., percent of the lung voxels below this threshold, integral of density values of all voxels below this threshold, and/or other density-based calculations). The quantification of emphysema can characterize an emphysematous disease state at a lobar level (generation 2), and/or at a segmental level (generation 3), and/or at a sub-segmental level (generation 4+). Additionally or alternatively, this −910 HU threshold can be used to characterize lobar lung volume and/or segmental lung volume. As described in further detail below, such lung metrics can be analyzed by additional software algorithm(s) to predict optimized treatment for particular lobe(s) and/or segment(s) of the lung.

As further shown in FIG. 23B, in some embodiments in which CT images from an inspiratory CT scan have a slice thickness of less than 3 mm (e.g., 1 mm), emphysema can be quantified or otherwise characterized by assessing lung voxels on an inspiratory CT scan having attenuation below −950 HU (e.g., percent of the lung voxels below this threshold, integral of density values of all voxels below this threshold, and/or other density-based calculations). The quantification of emphysema can characterize an emphysematous disease state at a lobar level and/or at a segmental level. Additionally or alternatively, this −950 HU threshold can be used to characterize lobar lung volume and/or segmental lung volume, and/or lack of perfusion at a lobar level and/or at a segmental level.

As another example, as shown in FIG. 23B, the first software algorithm can incorporate one or more voxel density thresholds associated with an expiratory CT scan. In some embodiments, air trapping can be quantified or otherwise characterized by assessing lung voxels on an expiratory CT scan having attenuation below −856 HU (e.g., percent of the lung voxels below this threshold, integral of density value of all voxels below this threshold, and/or other density-based calculations). Additionally or alternatively, in some embodiments the first software algorithm can include generating lung metrics characterizing one or more lung anatomical features (e.g., measurements of airways within the lung) from image data from an expiratory CT scan. For example, the first software algorithm can generate lung metrics from an expiratory CT scan including geometrical, physical, and/or mechanical properties of the airways, such as airway compliance, pleural pressure, airway diameter to wall thickness, and/or airway wall deformation. Such properties can be measured particularly at the segmental level, which (compared to such information determined solely at the lobar level) can provide more insight into the patient condition for use in determining patient candidacy for treatment, treatment planning, etc. Additionally or alternatively, the first software algorithm can generate lung metrics from an expiratory CT scan including characterization of lobar volume and/or segmental lung volume, and/or identification of collapsed airways (and their severity of collapse, such as airway diameter) that may be contributing to the disease state (e.g., hyperinflation and impairment of breathing).

It should be understood that the above-described density threshold values for analyzing CT scans are only examples, and that other suitable threshold values may additionally or alternatively be used to evaluate voxel density data and generate suitable lung metrics. For example, a density threshold value for an inspiratory CT scan can be a value ranging from −910 HU to −990 HU, or ranging from −950 HU to −990 HU, or ranging from −970 HU to −990 HU (e.g., −950 HU, −960 HU, −970 HU, −980 HU, or −990 HU).

The lung metrics produced by the first software algorithm can be provided to a second software algorithm. The second software algorithm can be a second machine learning algorithm that has been trained (e.g., via supervised learning) to analyze the lung metrics to determine whether the patient is a candidate for one or more treatments for the pulmonary disease. In some embodiments, the second software algorithm can additionally or alternatively include other suitable automated processes. For instance, the output of the second software algorithm can be the likelihood of success if the patient receives the treatment. The treatment can be an airway treatment for COPD, such as a pharmacological treatment (e.g., bronchodilators), an interventional treatment (e.g., vapor therapy, administration of a sealant, transbronchial fenestration, placement of an endobronchial coil, placement of an endobronchial valve, or placement of a minimal endobronchial reinforcement implant), or a combination thereof.

In some embodiments, the output of the second software algorithm is a predicted response of the patient to the treatment. The predicted response can include a prediction of any of the following: FEV (e.g., FEV₁), FVC, VC, IC, IC/TLC, functional residual capacity, TLC, diffusion capacity for carbon monoxide, RV/TLC, RV, segmental volume, mMRC score, SGRQ score (or the score for a subset of SGRQ questions), CAT score (or the score for a subset of CAT questions), 6-minute walk test results, cycle ergometry results, cardiopulmonary exercise testing (CPET) results, patient health metrics (e.g., heart rate, blood pressure, body mass index), patient exercise metrics (e.g., number of steps taken), patient visit metrics, quality of life metrics (e.g., ability to breathe), number of required implant removals, time to reintervention, durability of treatment, comorbidities, drug regimen, length of hospitalization, healthcare utilization, and/or cost; and/or a change (e.g., increase or decrease) in any of the above. Optionally, the predicted response can include a comparison of the response to endobronchial implant therapy with a minimal endobronchial reinforcement implant versus other treatment procedures, such as pharmacological treatments, interventional treatments (e.g., valves, coils, steam, hydrogel glue, thermal ablation, non-thermal ablation), surgical treatment, etc.

If the patient is predicted to have a favorable response to the treatment (e.g., to endobronchial implant therapy with a minimal endobronchial reinforcement implant), the method 2000 can include using a third software algorithm to generate a treatment plan for the patient. The third software algorithm can be a third machine learning algorithm that has been trained (e.g., via supervised learning) to analyze the lung metrics and/or predicted response to predict an optimized placement of one or more endobronchial implants to treat the patient's pulmonary disease. In some embodiments, the third software algorithm can additionally or alternatively include other suitable automated processes. The output of the third software algorithm can be a treatment plan. In embodiments where the treatment includes endobronchial implant therapy, the treatment plan can include any of the following information: implant placement location, number of implants, implant size, implant type, implant geometry, pathway to a target location for implant placement, and/or localized treatment solutions. For example, implant placement location may be based at least in part on location of dynamic airway collapse as observed in or otherwise determined from expiratory CT data, severity of pulmonary disease in the peripheral lung, the location of the pleural wall, and/or the location of lobar, segmental, and/or sub-segmental airways.

For example, in some embodiments, the third software algorithm can assist in the selection of one or more lobes and/or one or more airway segments and/or sub-segments for implant placement locations based on lung metrics (e.g., lung metrics provided by the first software algorithm). For example, in identifying suitable implant placement locations, the third software algorithm can target lobes with higher amounts of emphysema destruction (e.g., higher disease score) and with larger lobar volume. Additionally or alternatively, the third software algorithm can target airway segments with higher amounts of emphysema destruction (e.g., higher disease score) and with larger segment volume.

Additionally or alternatively, in some embodiments, the third software algorithm can analyze lung metrics relating to one or more fissures in the lung (e.g., lobar fissures and/or segmental fissures) to generate a treatment plan for the patient. The analyzed lung metrics can include, for example, identification of position, shape, and/or degree of completeness of fissures between lobes and/or between segments within lobes. In some embodiments, to optimize placement of a minimal endobronchial reinforcement implant, it may be advantageous to place the implant at target locations where the fissures are incomplete. Without being bound by any particular theory, it is believed that incomplete fissures allow for more communication of airflow between adjacent segments and/or adjacent lobes, thereby allowing release of trapped air from more segments (and/or lobes) with fewer endobronchial reinforcement implant(s). Accordingly, the third software algorithm can be configured to identify an endobronchial reinforcement implant placement location that is proximate one or more incomplete lobar and/or segmental fissures. In contrast, for some interventional treatments (e.g., placement of one-directional stent valves, which allow air to flow out of but not into overinflated portions of the lung), it may be advantageous to place the implant at target locations where fissures are complete.

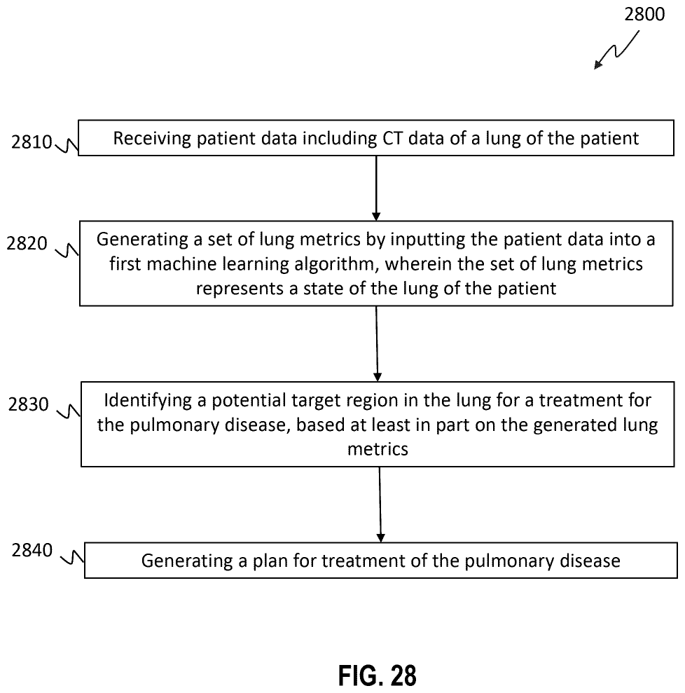

The method 2000 can be modified in many different ways. For example, any of the software algorithms illustrated in FIG. 20 can be combined with each other, subdivided into separate algorithms, and/or omitted altogether. For example, as shown in FIG. 28, a method 2800 can be similar to method 2000 except the method 2800 omits at least the second software algorithm (e.g., predicting response of the patient to the treatment). For example, method 2800 can include receiving patient data including CT data of a lung of the patient (block 2810), generating a set of lung metrics by inputting the patient data into a first machine learning algorithm (block 2820), identifying a potential target region in the lung for a treatment for the pulmonary disease based at least in part on the generated lung metrics (block 2830), and generating a plan for treatment of the pulmonary disease (block 2840). In some embodiments, block 2830 and block 2840 can incorporate or be similar to one or more aspects of the third software algorithm. In some embodiments, the first and second software algorithms are combined or replaced with a single software algorithm that directly predicts the patient response from the patient data. Optionally, the third software algorithm can generate a treatment plan directly from the patient data, without requiring the lung metrics and/or predicted response generated by the first and second software algorithms, respectively.

In some embodiments any of the outputs of the first software algorithm, the second software algorithm, and/or the third software algorithm in the method 2000 can be provided to a user in the form of a patient report. For example, the report can include any one or more of the lung metrics of interest generated by the first software algorithm, a response of the patient predicted by the second software algorithm, and/or a treatment plan generated by the third software algorithm. The report may include written summaries or descriptions of any of such outputs of the software algorithms of the method 2000, annotated diagrams, annotated images, and/or other media (e.g., videos). For example, the report can include an anatomical illustration representing at least a portion of a lung and/or an image (e.g., CT image) of at least a portion of a lung that is annotated with suitable information. Suitable annotation information can include airway measurements (e.g., lumen diameter, wall thickness, etc.) along one or more sections of an airway, disease score for selected regions such as lobe(s), segment(s), and/or sub-segment(s) of a lung, lobe(s), portions of the lung having a disease score exceeding a predetermined threshold, and/or proposed implant placement locations, for example. In some embodiments, such annotations may be descriptive (e.g., with text or numbers) and/or otherwise visualized such as with color coding or line weights (e.g., thicker lines to emphasize airway walls in lung segments or sub-segments of interest). For example, an illustration or image of the lung may include a map or other visualization of the airways in the lung, and an identification of lung lobe(s) or segment(s) having a disease score that exceeds a predetermined threshold (e.g., with color coding or line weights). As another example, an illustration or image of the lung may include a map or other visualization of the airways in the lung, and an identification of one or more proposed implant placement locations (e.g., with an outline of the implant, highlighting of airway walls in a targeted airway segment with color or line thickness, etc.). In some embodiments, the report may be communicated to a patient medical record (e.g., electronic medical record) for reference.

The method 2000 can be performed using any suitable system or device. In some embodiments, some or all of the processes of the method 2000 are implemented as computer-readable instructions (e.g., program code) that are configured to be executed by one or more processors of a computing device.

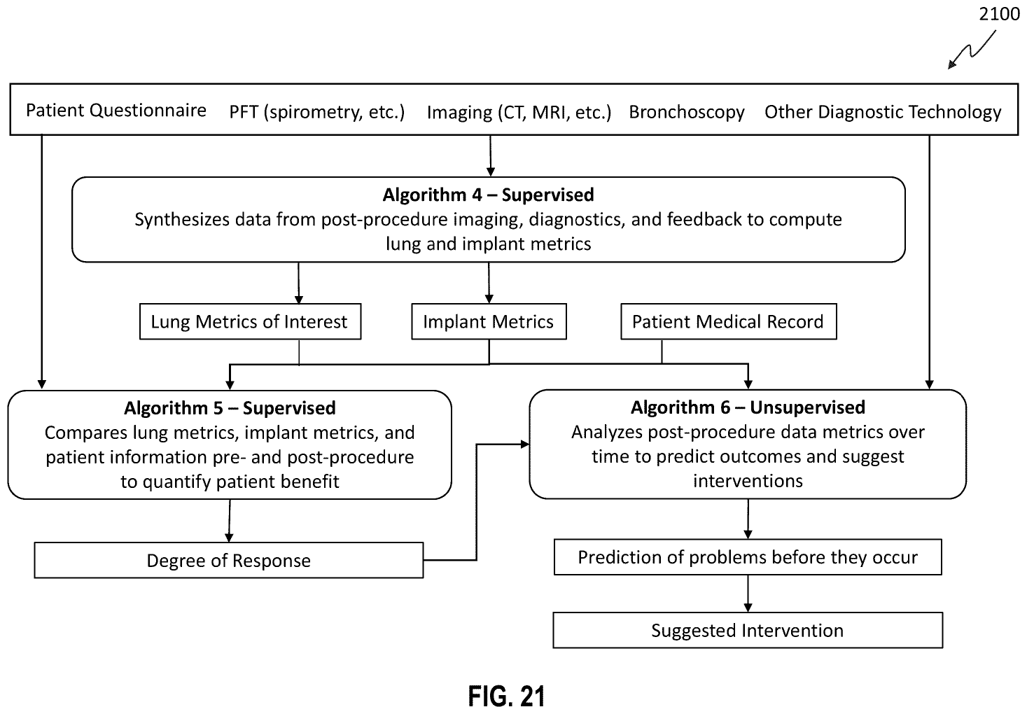

FIG. 21 is a flow diagram illustrating a method 2100 for evaluating a treatment outcome of a patient, in accordance with embodiments of the present technology. The method 2100 can be performed as part of the post-procedure phase 1906 of the workflow 1900 of FIG. 19. Although the method 2100 is described herein in connection with evaluating the outcome of endobronchial implant therapy, in other embodiments, the method 2100 can be modified for use in evaluating the outcomes of other types of airway treatments (e.g., pharmacological treatments, non-implant interventional treatments).

The method 2100 can involve receiving patient data, such as questionnaire information, medical record information, image data (e.g., CT data, MRI data, chest radiography data, fluoroscopy data, photographs), PFT data (e.g., spirometry data), bronchoscopy data, data from other diagnostic technologies, and/or any of the other patient data types described herein. Some or all of the patient data can be obtained at a plurality of time points, as discussed above with respect to FIG. 20. The method 2100 can implement one or more software algorithms that use the patient data to analyze whether the treatment procedure was successful and, optionally, propose additional procedures that may further improve the patient outcome.

The patient data (e.g., questionnaire information, PFT data, image data, bronchoscopy data, and/or data from other diagnostic technologies) can be provided to a fourth software algorithm. The fourth software algorithm can be a fourth machine learning algorithm that has been trained (e.g., via supervised learning) to synthesize the patient data to compute lung metrics and/or implant metrics. In some embodiments, the fourth software algorithm can additionally or alternatively include other suitable automated processes. For instance, the lung metrics can represent a state of the patient's lung after the placement of one or more endobronchial implants (e.g., minimal endobronchial reinforcement implants), such as any of the following: FEV (e.g., FEV₁), FVC, VC, IC, IC/TLC ratio, functional residual capacity, TLC, diffusion capacity for carbon monoxide, RV, RV/TLC ratio, CV, lobar and/or segmental tissue destruction, lobar and/or segmental air trapping, lobar and/or segmental fissure status, extent of lobar and/or segmental fissure completion, lobar and/or segmental ventilation, lung function, disease phenotype (e.g., homogeneity/heterogeneity of emphysema, type of emphysema (such as centriacinar emphysema, panacinar emphysema, or paraseptal emphysema), location of diseased portions of the lung), lobar volume, segmental volume, segment locations, diaphragm shape, tissue density, opacity, proximity of diseased portions to anatomical structures, proximity of disease portions to other medical devices, lumen inner diameter of bronchial sections, air flow mapping, collapsed airways, airway pressure, airway compliance, pleural pressure, airway diameter to wall thickness, airway wall deformation, vascular perfusion, parenchyma density, bullae, information that localizes disease in the lung, obstruction score, mucus score, degree of epithelialization, other implant-tissue interactions (e.g., granulation tissue, implant-induced airway deformation, airway tissue invagination into a lumen of the implant, etc.). The set of lung metrics can characterize the state of the lung at a single time point, and/or can characterize a change in the state of the lung over plurality of time points (e.g., before and after endobronchial implant therapy, before and after administration of a bronchodilator, before and during exercise). Additionally or alternatively, the lung metrics can be used to generate a disease “score” characterizing severity of pulmonary disease that may remain in one or more regions of the lung following treatment and/or representing an estimate of patient response to a treatment of the pulmonary disease. In some embodiments, the lung metrics can include one or more disease scores, where each disease score corresponds to a respective region of the lung (e.g., a particular lung, a particular lobe, a particular segment, a particular sub-segment). In some embodiments, the lung metrics can include a single disease score at least partially based on multiple “local” disease scores each corresponding to a respective region of the lung (e.g., a particular lung, a particular lobe, a particular segment, a particular sub-segment). For example, a single disease score can be an average value of the multiple local disease scores, or be based on any suitable calculation incorporating the multiple local disease scores. The method 2700 as shown in FIG. 27 for evaluating a patient using such a disease score lung metric (described above with respect to a pre-procedure evaluation) can additionally or alternatively be performed as a post-procedure evaluation.

The implant metrics can represent a state of each endobronchial implant after placement in the lung, such as any of the following characteristics: implant location, distance between a distal end of the implant and pleura, implant length, implant diameter at any one or more locations along the implant length, implant cross-sectional profile at any one or more locations along the implant length, implant integrity (e.g., implant breakage, deformation), indication of implant expansion and/or collapse (e.g., biasing of the implant in a longitudinal direction, or “pancaking”) such as pitch of implant loops or angle of implant loop profile relative to a longitudinal axis of the implant), implant position relative to other placed implant(s), movement of one or more implants between inspiration and expiration, occlusion of implant, and/or implant dislodgement. The implant metrics can represent the state of the implant at a single time point, or can represent a change in the state of the implant over a plurality of time points. For example, the fourth software algorithm identifying changes in implant properties from CT imaging taken over various time points may be helpful to understand impacts to patient benefit and/or implant interactions.

For example, in some embodiments, the fourth software algorithm can analyze a follow up expiratory CT scan to generate lung metrics including geometrical, physical, and/or mechanical properties of the airways, such as airway compliance, pleural pressure, airway diameter to wall thickness, and/or airway wall deformation. Such properties can be measured particularly at the segmental level, which (compared to such information determined solely at the lobar level) can provide more insight into the patient condition for use in determining a response of the patient to treatment. Additionally or alternatively, the fourth software algorithm can generate lung metrics including identification of any remaining collapsed airways (and their severity of collapse, such as airway diameter). Furthermore, in some embodiments the fourth software algorithm can generate implant metrics characterizing state of the placed implant from the expiratory CT scan, such as any of those described above. Additionally or alternatively, the fourth software algorithm can include other suitable quantitative CT (QCT) techniques, such as those described in further detail herein. Identification of geometric and/or positional changes of the implant over time as well as changes to lung anatomy such as lobe volume, diaphragm shape, density, opacity, and/or segment location could be extracted to draw connections between patient anatomy, implant orientation, and treatment results.

In some embodiments, the method 2100 uses a fifth software algorithm that analyzes the patient data (e.g., questionnaire information, PFT data, image data, bronchoscopy data, medical record information, and/or data from other diagnostic technologies), lung metrics, and/or implant metrics to determine a response of the patient to endobronchial implant therapy. The fifth software algorithm can be a fifth machine learning algorithm that has been trained (e.g., via supervised learning) to compare the lung metrics, implant metrics, and/or patient data pre- and post-procedure to quantify the benefits to the patient. In some embodiments, the fifth software algorithm can additionally or alternatively include other suitable automated processes. The output of the fifth software algorithm can be indicators of the degree of patient response such as FEV (e.g., FEV₁), FVC, VC, IC, IC/TLC, functional residual capacity, TLC, diffusion capacity for carbon monoxide, RV/TLC, RV, segmental volume, mMRC score, SGRQ score or a subset thereof, CAT score or a subset thereof, 6-minute walk test results, cycle ergometry results, CPET results, patient health metrics (e.g., heart rate, blood pressure, body mass index), patient exercise metrics (e.g., number of steps taken), patient visit metrics, quality of life metrics (e.g., ability to breathe), number of required implant removals, time to reintervention, durability of treatment, comorbidities, drug regimen, length of hospitalization, healthcare utilization, and/or cost; and/or a change (e.g., increase or decrease) in any of the above.

In some embodiments, for example, the fifth software algorithm can include analyzing pre- and post-procedure CT scans, which may provide additional insight to the effect and/or efficacy of treatment. For example, as shown in FIG. 24, the fifth software algorithm can analyze changes in lung volume, lobar volume, segmental volume, fissure position(s) (e.g., status and/or extent of completeness), diaphragm shape, central airway shape (e.g., collapsed central airways due to hyperinflation compared to normal central airways due to release of trapped air post-procedure), etc. based on information derived from CT scans (e.g., evaluation of voxel density, etc.). Additionally or alternatively, the fifth software algorithm can include other suitable quantitative CT (QCT) techniques, such as those described in further detail herein.

In some embodiments, the method 2100 uses a sixth software algorithm that analyzes the patient data (e.g., questionnaire information, PFT data, image data, bronchoscopy data, medical record information, and/or data from other diagnostic technologies), lung metrics, implant metrics, and/or determined response to predict patient outcome after endobronchial implant therapy. The sixth software algorithm can be a sixth machine learning algorithm that has been trained (e.g., via unsupervised learning) to analyze post-procedure data metrics over time to predict future outcomes. In some embodiments, the sixth software algorithm can additionally or alternatively include other suitable automated processes. For example, the predicted outcomes can include whether the patient's prognosis is likely to improve, remain stable, or deteriorate over time. As another example, the predicted outcomes can include a prediction of a post-procedure issue, such as copious mucus, excessive granulation tissue, excessive fibrosis, implant collapse, implant migration, implant failure, implant invagination, implant occlusion, implant expectoration, inadequate lung function, pneumothorax, infection, pneumonia, and/or hospitalization.

In some embodiments, the sixth software algorithm determines one or more interventions to prevent, mitigate, or otherwise address the predicted issue, such as cleanup bronchoscopy, retrieval and/or removal of one or more implants, replacement of one or more implants, repositioning of one or more implants, dilation of one or more implants, placement of one or more additional implants (e.g., placing one or more additional implants in other lobes or a contralateral lung), consultation with healthcare professionals, and/or additional treatment procedures (e.g., medication, surgery, other medical devices). For instance, cleanup bronchoscopy at scheduled intervals can be recommended if copious mucus is predicted. Implant removal can be recommended if excess granulation tissue and/or fibrosis is predicted. Balloon dilatation can be recommended if implant collapse is predicted. Placement of an additional implant can be recommended if inadequate improvement in FEV₁ is predicted. Implant removal or replacement can be recommended if implant failure is predicted. Preventative doctor visits can be recommended if hospitalization is predicted.

The method 2100 can be modified in many different ways. For example, any of the software algorithms illustrated in FIG. 21 can be combined with each other, subdivided into separate algorithms, and/or omitted altogether. Optionally, the fifth software algorithm can determine the patient response directly from the patient data, without requiring the lung metrics and/or implant metrics generated by the fourth software algorithm, respectively. Similarly, the sixth software algorithm can predict the patient outcome directly from the patient data, without requiring the lung metrics and/or implant metrics generated by the fourth software algorithm, and/or without the patient response determined by the fifth software algorithm.

Similar to that described with respect to the method 2000, in some embodiments any of the outputs of the fourth software algorithm, the fifth software algorithm, and/or the sixth software algorithm in the method 2100 can be provided to a user in the form of a patient report. For example, the report can include any one or more of the lung and implant metrics of interest generated by the fourth software algorithm, a patient response to endobronchial implant therapy (e.g., quantified patient benefit) generated by the fifth software algorithm, and/or predicted future patient outcome(s) and/or proposed interventions generated by the third software algorithm. The report may include written summaries or descriptions of any of such outputs of the software algorithms of the method 2100, annotated diagrams, annotated images, and/or other media (e.g., videos). For example, the report can include an anatomical illustration representing at least a portion of a lung and/or an image (e.g., CT image) of at least a portion of a treated lung and/or implant placed in the treated lung that is annotated with suitable information. Suitable annotation information can include airway measurements (e.g., lumen diameter, wall thickness, etc.) along one or more sections of an airway, disease score for selected regions such as lobe(s), segment(s), and/or sub-segment(s) of a lung, lobe(s), portions of the lung having a disease score exceeding a predetermined threshold, and/or implant placement location(s), for example. In some embodiments, such annotations may be descriptive (e.g., with text or numbers) and/or otherwise visualized such as with color coding or line weights (e.g., thicker lines to emphasize airway walls in lung segments of interest). For example, an illustration or image of the lung may include a map or other visualization of the airways in the lung, and an identification of lung lobe(s), segment(s), or sub-segment(s) having a disease score that exceeds a predetermined threshold (e.g., with color coding or line weights). As another example, an illustration or image of the lung may include a map or other visualization of the airways in the lung, and an identification of one or more implant locations (e.g., with an outline of the implant, highlighting of airway walls in an airway segment with color or line thickness, etc.). As another example, the report can include access to a virtual bronchoscopy based on a reconstructed 3D model of the treated lung and placed implant, as described herein. In some embodiments, the report may be communicated to a patient medical record (e.g., electronic medical record) for reference.

The method 2100 can be performed using any suitable system or device. In some embodiments, some or all of the processes of the method 2100 are implemented as computer-readable instructions (e.g., program code) that are configured to be executed by one or more processors of a computing device.

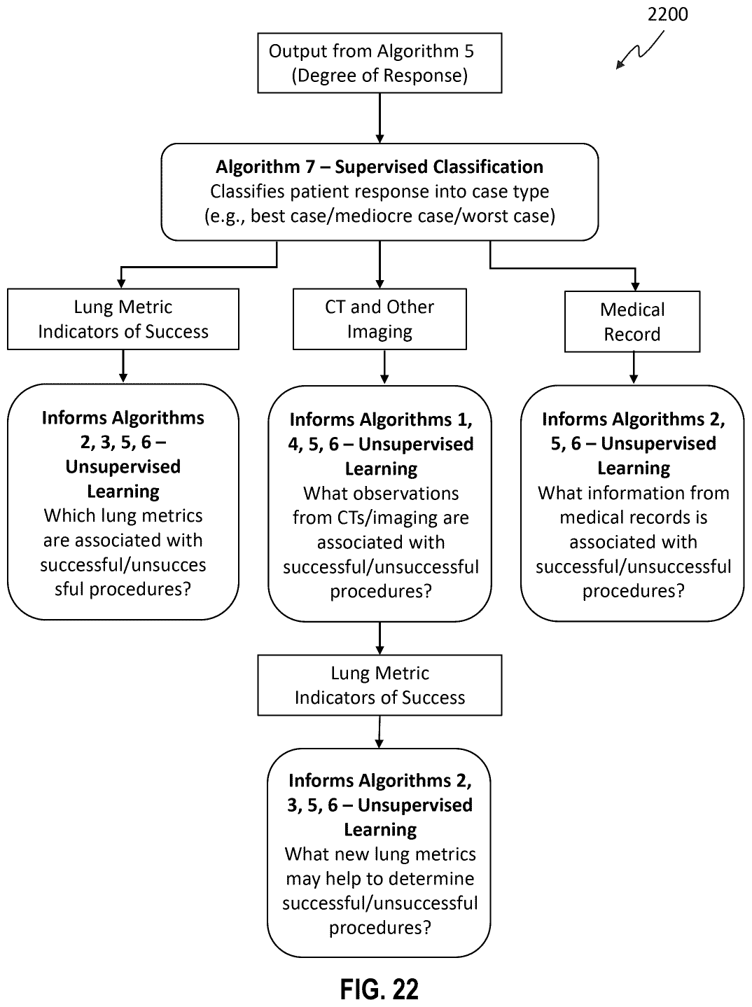

FIG. 22 is a flow diagram illustrating a method 2200 for updating the software algorithms of FIGS. 20 and 21, in accordance with embodiments of the present technology. The method 2200 can involve obtaining data regarding patient responses to endobronchial implant therapy from a plurality of different patients. The data may be generated by the fifth software algorithm of the method 2100 of FIG. 21. The patient response data can be provided to a seventh software algorithm. The seventh software algorithm can be a seventh machine learning algorithm that is trained (e.g., via supervised learning techniques) to classify the patient response to treatment into various categories, such as “best case,” “worst case,” “mediocre case,” etc. In some embodiments, the seventh software algorithm can additionally or alternatively include other suitable automated processes. The outputs of the seventh software algorithm can be correlated to other types of data, such as indicators of treatment success (e.g., lung metrics), image data (e.g., CT data), and/or other types of patient data (e.g., medical record information). These correlations can be used to update at least some of the software algorithms of FIGS. 20 and 21.

For example, correlations between patient responses and lung metrics (and/or other indicators of treatment success) can be used to update the second software algorithm (FIG. 20), third software algorithm (FIG. 20), fifth software algorithm (FIG. 21), and/or sixth software algorithm (FIG. 21). In embodiments where these software algorithms are or include machine learning algorithms, the correlations can be used as training data for the machine algorithms (e.g., for unsupervised learning). The correlations can inform the algorithms on which lung metrics and/or other indicators of treatment success are associated with successful treatment procedures versus unsuccessful procedures.

As another example, correlations between patient responses and image data (e.g., CT data) can be used to update the first software algorithm (FIG. 20), fourth software algorithm (FIG. 21), fifth software algorithm (FIG. 21), and/or sixth software algorithm (FIG. 21). In embodiments where these software algorithms are or include machine learning algorithms, the correlations can be used as training data for the machine algorithms (e.g., for unsupervised learning). The correlations can inform the algorithms on which observations from CT data and/or other image data are associated with successful treatment procedures versus unsuccessful procedures. Optionally, observations from CT data and/or other image data that are associated with successful and/or unsuccessful treatment procedures can be used to update (e.g., train) the second software algorithm (FIG. 20), third software algorithm (FIG. 20), fifth software algorithm (FIG. 21), and/or sixth software algorithm (FIG. 21) to determine new lung metrics and/or other indicators of treatment success that may be helpful in predicting the success or failure of a treatment procedure.

In a further example, correlations between patient responses and medical record information can be used to update the second software algorithm (FIG. 20), fifth software algorithm (FIG. 21), and/or sixth software algorithm (FIG. 21). In embodiments where these software algorithms are or include machine learning algorithms, the correlations can be used as training data for the machine algorithms (e.g., for unsupervised learning). The correlations can inform the algorithms on what information from patient medical records are associated with successful treatment procedures versus unsuccessful procedures.

Any of the software algorithms described herein (e.g., Algorithms 1-7 of FIGS. 20-22) can be updated and/or refined based on historical and/or repository patient data. This historical patient data may be sourced from a database of previous patients, which could include data from the same patient at earlier time points as well as data of other patients. This historical patient data may also be sourced from a repository that may include patients with lung diseases (e.g., GOLD III COPD, GOLD IV COPD) with or without interventions, such as endobronchial implants (e.g., minimal endobronchial reinforcement implants, valves, coils), vapor therapy, or pharmacological treatments (e.g., inhaled bronchodilators). For example, the software algorithms can be updated for predictive power using hierarchical Bayesian modeling, with the prior probability in the Bayesian scheme derived from historical or repository patient data.

### V. Platform-Agnostic Quantitative CT (QCT)

As described in further detail herein, imaging data from CT scans may be analyzed by one or more software algorithms (e.g., machine learning algorithms) to obtain lung metrics and/or implant metrics. To generate lung metrics and/or implant metrics, methods and systems in accordance with the present technology can include one or more software algorithms (e.g., machine learning algorithm or other automated algorithm) to identify and characterize quantitative image features. In order for a quantitative image feature to effectively serve as a biomarker for disease diagnosis and/or assessment of implant therapy (e.g., placement of an endobronchial reinforcement implant), the quantitative image feature must be reproducible. However, scanning techniques, parameters, and other scanner algorithms (also referred to collectively herein as “CT scan parameters”) can have a large impact on the reproducibility of QCT outputs. Different CT scanners (e.g., different scanner manufacturers) may vary and any particular CT scanner may also be operated with various scan parameters, thereby affecting the results of QCT analysis. For example, the radiation dose applied during scanning, and/or the reconstruction algorithm used to generate tomographic images from acquired x-ray projection data, may affect the results of QCT analysis. These and further examples of CT scan parameters that can impact QCT analysis are listed below in Table 1.

TABLE 1
CT scan parameters

| | |
|---|---|
| CT Scan Parameter | Description |

1. Scan acquisition

| | |
|---|---|
| CT localizer radiograph | Type of scanned projection radiograph acquired to allow the |
| | user to prescribe the start and end locations of the scan range |
| Axial scan mode | Data acquisition process while the patient table remains |
| | stationary; the table position may be incremented between x- |
| | ray exposures to collect data over a longer z-axis range |
| Helical or spiral scan mode | Data acquisition process while the patient table is |
| | continuously moving along the z-axis |
| Dynamic scan mode - single | Data acquisition process at multiple time points over the same |
| detector width | anatomic location(s) while the patient table remains |
| | stationary; x-ray exposure can be continuous or intermittent |
| Dynamic scan mode - | Data acquisition process at multiple time points over the same |
| multiple detectors widths | anatomic location(s) while the patient table cycles back and |
| | forth between designated start and end locations in order |
| | image a region wider than the detector |
| Interventional CT - | Data acquisition process for intermittent x-ray exposures |
| Intermittent x-ray exposures | (e.g., to reduce total radiation exposure) |
| Interventional CT - | Data acquisition process for continuous x-ray exposures |
| Continuous x-ray exposures | |
| Table increment - axial scan | Table increment per 360 degree rotation of the x-ray tube in |
| mode | axial scan mode |
| Table feed - helical scan | Table feed per 360 degree rotation of the x-ray tube in helical |
| mode | scan mode |
| Field of measurement | Diameter of the circular region within the scan plane over |
| | which projection data are collected. Nominally equal to the |
| | diameter of the primary beam at isocenter in the axial plane. |
| Automatic exposure control | Algorithm for automatically adjusting the x-ray tube current |
| (AEC) | to the overall patient size to achieve a specified level of image |
| | quality |
| Tube current | Current/number of electrons accelerated across an x-ray tube |
| | per unit time, expressed in milliampere (mA) |
| Tube current-time product | The product of tube current and exposure time perf rotation, |
| | expressed in milliampere*seconds (mAs). In axial scan mode, |
| | this is equal to tube current * (scan angle ÷ 360) * rotation |
| | time. In helical scan mode, this is equal to tube current * |
| | rotation time. |
| Effective tube current-time | In helical scan mode, this is the product of tube current and |
| product | rotation time, expressed in units of milliampere*seconds |
| | (mAs) ÷ pitch) |
| Tube potential | Electric potential applicated across an x-ray tube to accelerate |
| | electronics, expressed in kilovolts (kV) |
| Pitch | Unitless parameter used to describe the table travel during |
| | helical CT, equal to table travel per gantry rotation ÷ total |
| | nominal beam width |

2. Dose modulation and reduction

| | |
|---|---|
| Automatic exposure control | Existence of scanner feature that automatically adapts the x- |
| (AEC) | ray tube current to the overall patient size to achieve a |
| | specified level of image quality |
| Angular tube current | Algorithm for automatically adjusting the tube current at |
| modulation | different x-ray beam projection angles in each slice location |
| Longitudinal tube current | Algorithm for automatically adjusting the mean tube current |
| modulation | per rotation in each slice location |
| Image quality reference | Image quality reference for automatic exposure control |
| parameter for AEC | (AEC) |
| Automatic tube voltage | Algorithm for automatically selecting tube voltage |
| selection | |
| Organ based tube current | Algorithm for automatically modulating tube current based on |
| modulation | target organ type |

3. Dual Energy CT Imaging

| | |
|---|---|
| Dual-energy computer | Algorithm for dedicated CT imaging technology acquiring |
| tomography (dual energy CT | two data sets from two distinct photon energy levels with a |
| imaging) | minimal time interval. |
| Basis materials (dual energy | At a given energy, materials have unique photoelectric and |
| CT imaging) | Compton effects. Such materials, known as basis materials, |
| | must have sufficiently different attenuation characteristics to |
| | be used for material decomposition |
| Dual-energy bone removal | Algorithm for a dual-energy CT application to remove bony |
| (dual energy CT imaging) | structures in angiography dual-energy CT images for vascular |
| | structure enhancement |
| Effective atomic number | A description of the average atomic number for a |
| (Zeff) (dual energy CT | heterogenous material |
| imaging) | |
| Electron density images (dual | CT images representing the density of the material electrons. |
| energy CT imaging) | Its value usually is normalized to water electron density |
| Iodine map (dual energy CT | Multi-energy CT material selective images of iodine |
| imaging) | equivalent materials with water equivalent materials removed |
| Mixed or blended images | Algorithm for blending low and high energy image sets into |
| (dual energy CT imaging) | one new image set |
| Mono-E curves (dual energy | A plot of a curve representing the variation of CT number of a |
| CT imaging) | region of interest across multiple monoenergetic energies |
| Virtual mono-energetic (dual | Synthetic images mimicking the appearance of images from a |
| energy CT imaging) | true mono-energetic photon source. These images are |
| | generated from the energy-independent information available |
| | during material decomposition. |
| Virtual non-calcium (dual | Multi-energy CT material selective images with calcium |
| energy CT imaging) | equivalent materials removed. It is usually used to visualize |
| | bone marrow |
| Virtual non-contrast (dual | Material selective images with the contrast (iodine) removed. |
| energy CT imaging) | Soft-tissue, fat and iodine are the base materials |

4. Multi-slice detector geometry

| | |
|---|---|
| Multi-slice detector array | Array design of multi-sliced detector (e.g., dimensions, fixed |
| design | vs. adaptive) |
| Detector configuration | Configuration type of individual detectors |

5. Image reconstruction

| | |
|---|---|
| Pre-scan acquisition | Algorithm for prescribing the reconstruction parameters prior |
| reconstruction parameter | to scan acquisition |
| definition | |
| Post-scan acquisition | Algorithm for prescribing the reconstruction parameters after |
| reconstruction parameter | scan acquisition |
| definition | |
| Kernal/filter for image | Reconstruction algorithm that determines sharpness or |
| sharpness or smoothness | smoothness of image in the axial plane |
| Method for helical | Helical interpolation options to achieve a wider or narrower |
| interpolation | section sensitivity profile |
| Slice thickness | Nominal width of reconstructed image along z-axis |
| Slice interval | Distance between two consecutive reconstructed images |
| Fast scan | Algorithm for fast but lower-quality reconstructed images for |
| | rapid review of entire exam |

6. Contrast media

| | |
|---|---|
| Contrast agent type | Type of contrast agent administered to patient prior to scan |
| Contrast agent dosage | Dosage of contrast agent administered to patient prior to scan |
| Contrast agent administration | Method of administering contrast agent to patient prior to |
| modality | scan |
| Bolus tracking | Algorithm to automatically initiated a prescribed axial, |
| | helical, or dynamic scan when a threshold level of contrast |
| | enhancement is reached at a specified region of interest |
| Test bolus | Scan mode used to measure the contrast transit time using a |
| | small injection of contrast media |
| Threshold | CT number (HU) where bolus tracking tool will trigger the |
| | system to begin the scan |
| Monitoring delay | Time from injection to the start of enhancement monitoring |
| | scans prior to scan acquisition |
| Monitoring interval | Time between consecutive enhancement monitoring scans |
| | prior to scan acquisition |
| Scan delay | Time from when threshold is reached and prescribed axial, |
| | helical, or dynamic scan acquisition begins |

7. Multi-planar formats and 3D processing

| | |
|---|---|
| Oblique plane | Algorithm for reformatting image at an oblique plane (not an |
| | axial, coronal, or sagittal plane) |
| Multi-angle image saving | Algorithm for saving images at various viewing angles about |
| | a volume or surface rendered object |
| Multi-planar image saving | Algorithm for saving images at various planes through a |
| | volume |
| Surface object rendering | Algorithm for generating a surface-rendered object |
| Volume object rendering | Algorithm for generating a volume-rendered object |

In some embodiments, it may be advantageous to compensate for variance in QCT results due to CT scan parameters, such as by applying at least one correction factor. The correction factors can function to quantify the impact of differences in one or more CT scan parameters on resulting QCT analysis. Generally, such correction factors can be used to obtain a normalized (e.g., standardized) result to more effectively assess lung metrics and/or implant metrics, such as in a more objective manner that is CT platform-agnostic. For example, correction factors can be used to normalize the density of voxels associated with a particular x-ray attenuation threshold (e.g., −950 HU, −910 HU, etc.) in CT data. A single correction factor can be associated with a single respective CT scan parameter, or can be associated with multiple CT scan parameters used during a CT scan in combination. Furthermore, compensation for variance in a particular CT scan can involve the use of a single correction factor, or can involve the use of multiple correction factors.

In some embodiments, the correction factors associated with one or more particular CT scan parameters may be determined empirically. For example, CT scans of a phantom (or multiple phantoms) of known density can be repeatedly acquired under different predetermined imaging conditions (e.g., sets of known CT scan parameters). The effect on voxel density (and/or QCT analysis results) caused by change in any one particular CT scan parameter can be empirically determined by repeatedly acquiring CT scans of the phantom, while modulating the CT scan parameter in a known manner across different CT scans. For example, to assess the effect on voxel density caused by change in tube voltage, the tube voltage can be incrementally adjusted by a known amount between a series of CT scans of the phantom. The relationship between the CT scan parameter (and/or changes thereof) and resulting voxel density can be empirically determined (e.g., described by a formula). Additional CT scan parameters may be similarly individually modulated in a known manner to determine the relationship between other CT scan parameters (and/or changes thereof) and resulting voxel density.

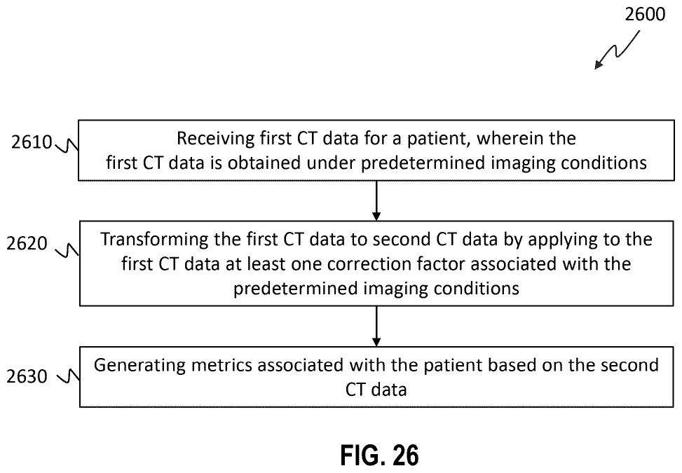

FIG. 26 is a flow diagram illustrating a method 2600 of normalizing one or more QCT results, such as lung metrics and/or implant metrics, for a patient (e.g., a patient having or suspected of having a pulmonary disease). The method 2600 can be incorporated in other methods described herein, such as in the method 1900 (e.g., to generate lung metrics and/or implant metrics of interest in a pre-procedure phase, a peri-procedure phase, and/or a post-procedure phase), in the method 2000 (e.g., to generate lung metrics of interest), method 2100 (e.g., to generate lung metrics of interest and/or implant metrics of interest) and/or method 2200. As shown in FIG. 26, the method 2600 can include receiving first CT data for a patient where the first CT data is obtained under predetermined imaging conditions (block 2610), transforming the first CT data to second CT data by applying at least one correction factor associated with the predetermined imaging conditions (block 2620), and generating metrics associated with the patient based on the second CT data (block 2630). Similar to that described above, the one or more correction factors applied to CT data can function to quantify the impact of the known imaging conditions such as CT scan parameters. Accordingly, in some variations, the metrics (e.g., lung metrics and/or implant metrics) generated in block 2630 can be more objective and CT platform-agnostic.

Receiving first CT data for a patient in block 2610 functions to obtain initial image data for a patient, taken under imaging conditions that are predetermined and/or otherwise known. The CT data can include inspiratory CT data (e.g., obtained at the end of full inspiration) and/or expiratory CT data (e.g., obtained at the end of forced expiration). The CT data can be generated through a CT scan acquisition process in a pre-procedure phase, a peri-procedure phase, and/or a post-procedure phase. The predetermined imaging conditions can include any one or more of the CT scan parameters listed in Table 1 (e.g., slice thickness, slice interval, tube potential, pitch, tube current, reconstruction algorithm, existence of any contrast agent administered to patient, contrast agent type/dosage, contrast agent administration modality, etc.). While these imaging conditions under which a scan is acquired are predetermined, each CT scan parameter may not be necessarily concretely known or identifiable. For example, some CT scan parameters (e.g., reconstruction algorithm and/or other algorithms used to determine fissure integrity) may be simply associated with a particular CT scanning machine design or CT vendor operating in a manner typical to that machine design or vendor. Other CT scan parameters (e.g., slice thickness, slice interval) may be both predetermined and known.

Transforming the first CT data to second CT data in block 2620 functions to convert the first CT data to a normalized (e.g., standardized) dataset from which more objective metrics can be analyzed (e.g., in block 2630). For example, the second CT data can include voxel density that are agnostic to differences in CT scan parameters (e.g., scan acquisition parameters, dose modulation parameters, image reconstruction algorithms, contrast media administration, etc.) and/or other variations such as CT scanner model and/or particular CT vendor providing CT imaging services. One or more correction factors can be applied to the first CT data to obtain the second, more normalized CT data. As described above, a single correction factor can be associated with a single respective CT scan parameter, or can be associated with multiple CT scan parameters used during a CT scan in combination.

In some embodiments, a correction factor can be associated with a group of multiple related CT scan parameters. For example, a correction factor can be associated with some or all of the CT scan parameters relating to scan acquisition as listed in Table 1, or including some or all of the CT scan parameters relating to dose modulation as listed in Table 1, or including some or all of the CT scan parameters relating to image reconstruction as listed in Table 1, or including some or all of the CT scan parameters relating to contrast media as listed in Table 1). In some embodiments, a correction factor can be associated with multiple groups of related CT scan parameters. Additionally or alternatively, in some embodiments, a correction factor can be associated with a particular CT scanning machine design (e.g., scanner model) and/or a particular vendor providing CT imaging services.

Generating metrics associated with the patient based on the second CT data in block 2630 functions to generate metrics (e.g., lung metrics and/or implant metrics) for use in further analysis. In some embodiments, such metrics are generated from the second CT data using one or more software algorithms, such as one or more trained machine learning algorithms and/or other automated algorithm. For example, any of the suitable software algorithms described above with respect to methods 1900, 2000, 2100, and/or 2200 can be used to generate lung metrics and/or implant metrics from the second CT data (e.g., the first software algorithm with respect to method 2000, the fourth software algorithm with respect to method 2100, etc.).

The lung metrics can include any of the lung metrics described herein, such as those described with respect to methods 1900, 2000, 2100, and/or 2200. For example, the lung metrics can represent a state of the patient's lung after the placement of one or more endobronchial implants (e.g., minimal endobronchial reinforcement implants), such as any of the following: FEV (e.g., FEV₁), FVC, VC, IC, IC/TLC ratio, functional residual capacity, TLC, diffusion capacity for carbon monoxide, RV, RV/TLC ratio, CV, lobar and/or segmental tissue destruction, lobar and/or segmental air trapping, lobar and/or segmental fissure status, extent of lobar and/or segmental fissure completion, lobar and/or segmental ventilation, lung function, disease phenotype (e.g., homogeneity/heterogeneity of emphysema, type of emphysema (such as centriacinar emphysema, panacinar emphysema, or paraseptal emphysema), location of diseased portions of the lung), lobar volume, segmental volume, segment locations, diaphragm shape, tissue density, opacity, proximity of diseased portions to anatomical structures, proximity of disease portions to other medical devices, lumen inner diameter of bronchial sections, air flow mapping, collapsed airways, airway pressure, airway compliance, pleural pressure, airway diameter to wall thickness, airway wall deformation, vascular perfusion, parenchyma density, bullae, information that localizes disease in the lung, obstruction score, mucus score, degree of epithelialization, other implant-tissue interactions (e.g., granulation tissue, implant-induced airway deformation, airway tissue invagination into a lumen of the implant, etc.). The set of lung metrics can characterize the state of the lung at a single time point, and/or can characterize a change in the state of the lung over plurality of time points (e.g., before and after endobronchial implant therapy, before and after administration of a bronchodilator, before and during exercise). Additionally or alternatively, the lung metrics can be used to generate a disease “score” characterizing severity of pulmonary disease that may remain in one or more regions of the lung following treatment. The lung metrics can include one or more disease scores, where each disease score characterizes severity of pulmonary disease in a respective region of the lung (e.g., a particular lung, a particular lobe, a particular segment, a particular sub-segment).

The implant metrics can include any of the implant metrics described herein, such as those described with respect to methods 1900, 2000, 2100, and/or 2200. For example, the implant metrics can represent a state of each endobronchial implant after placement in the lung, such as any of the following characteristics: implant location, distance between a distal end of the implant and pleura, implant length, implant diameter at any one or more locations along the implant length, implant cross-sectional profile at any one or more locations along the implant length, implant integrity (e.g., implant breakage, deformation), indication of implant expansion and/or collapse (e.g., biasing of the implant in a longitudinal direction, or “pancaking”) such as pitch of implant loops or angle of implant loop profile relative to a longitudinal axis of the implant), implant position relative to other placed implant(s), movement of one or more implants between inspiration and expiration, occlusion of implant, invagination, and/or implant dislodgement. The implant metrics can represent the state of the implant at a single time point, or can represent a change in the state of the implant over a plurality of time points.

In some embodiments, various methods in accordance with the present technology can further include analyzing the generated metrics associated with the patient based on the second CT data. Such analysis can be performed with one or more software algorithms, such as a trained machine learning algorithm or other automated algorithm. For example, lung metrics based on the second CT data can be analyzed in a patient selection process, a procedure planning process, an outcome assessment process, and/or an intervention recommendation process. As another example, implant metrics based on the second CT data can be analyzed in an outcome assessment process and/or an intervention recommendation process.

In some embodiments, any of the suitable software algorithms described above with respect to methods 1900, 2000, 2100, and/or 2200 can be used to analyze the metrics generated based on the second CT data. For example, lung metrics based on the second CT data can be analyzed using the second software algorithm described with respect to method 2000 to determine whether a patient is a candidate for treatment (e.g., likelihood of treatment success), and/or using the third software algorithm described with respect to method 2000 to predict an optimized treatment plan (e.g., optimized implant placement). Additionally or alternatively, lung metrics and/or implant metrics based on the second CT data can be analyzed using the fifth software algorithm with respect to method 2100 to quantify patient benefit following implant placement, and/or using the sixth software algorithm to predict future outcomes and/or suggest interventions following implant placement.

### CONCLUSION

Although many of the embodiments are described above with respect to systems, devices, and methods for treating COPD and emphysema, the technology is applicable to other applications and/or other approaches, such as identifying and/or treating tracheobronchomalacia (TBM), excessive dynamic airway collapse (EDAC), or benign prostatic hyperplasia (BPH). Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-28.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method for planning a treatment for a patient having a pulmonary disease, the method comprising:
receiving patient data including computed tomography (CT) data of a lung of the patient;
generating a set of lung metrics by inputting the patient data into a first machine learning algorithm, wherein the set of lung metrics represents a state of the lung of the patient;
predicting a response of the patient to a treatment for the pulmonary disease by inputting the set of lung metrics into a second machine learning algorithm; and
evaluating whether the patient is a candidate for the treatment for the pulmonary disease, based on the predicted response.

2. The method of claim 1, wherein the patient data comprises one or more of the following: questionnaire information, medical record information, magnetic resonance imaging (MRI) data, single-photon emission computed tomography (SPECT) data, bronchoscopy data, ventilation-perfusion data, pulmonary function test data, chest radiography data, fluoroscopy data, photographs, or sensor data.

3. The method of claim 1 or 2, wherein the CT data comprises expiratory CT data.

4. The method of claim 3, wherein the CT data comprises inspiratory CT data.

5. The method of any one of claims 1-4, wherein the patient data comprises data obtained at a plurality of different time points.

6. The method of any one of claims 1-5, wherein the set of lung metrics correlates to whether the patient has at least one of chronic obstructive pulmonary disease (COPD), severe emphysema, or severe emphysema with hyperinflation.

7. The method of any one of claims 1-6, wherein the set of lung metrics characterizes one or more lung parameters, the one or more lung parameters comprising one or more of the following: forced expiratory volume in 1 second, forced vital capacity, vital capacity, inspiratory capacity, inspiratory capacity/total lung capacity ratio, functional residual capacity, total lung capacity, diffusion capacity for carbon monoxide, residual volume, residual volume/total lung capacity ratio, closing volume, lobar and/or segmental tissue destruction, lobar and/or segmental air trapping, lobar and/or segmental fissure status, extent of lobar and/or segmental fissure completion, lobar and/or segmental ventilation, lung function, homogeneity/heterogeneity of lobar and/or segmental emphysema, emphysema type, locations of diseased portions of the lung, lobar volume, segmental volume, segmental locations, diaphragm shape, tissue density, opacity, proximity of diseased portions to anatomical structures, proximity of diseased portions to other medical devices, lumen inner diameter of bronchial sections, air flow mapping, collapsed airways, airway pressure, airway compliance, pleural pressure, airway diameter to wall thickness, airway wall deformation, vascular perfusion, parenchyma density, bullae, information that localizes disease in the lung, obstruction score, mucus score, or degree of epithelialization.

8. The method of any one of claims 1-7, wherein the set of lung metrics comprises at least one disease score characterizing severity of pulmonary disease in the patient.

9. The method of claim 8, wherein the at least one disease score represents a predictor of patient response to the treatment of the pulmonary disease.

10. The method of claim 8 or 9, wherein the set of lung metrics comprises multiple disease scores each corresponding to a respective lobar, segmental, or sub-segmental region of the lung of the patient.

11. The method of claim 8 or 9, wherein the set of lung metrics comprises a single disease score based on multiple local disease scores each corresponding to a respective lobar, segmental, or sub-segmental region of the lung of the patient.

12. The method of claim 11, wherein the single disease score is an average of the multiple local disease scores.

13. The method of any one of claims 8-12, wherein the at least one disease score represents an extent of at least one of air trapping or hyperinflation in the lung of the patient.

14. The method of any one of claims 7-13, wherein the set of lung metrics characterizes a change in at least one of the one or more lung parameters over a plurality of time points.

15. The method of claim 14, wherein the plurality of time points comprise two or more of the following: before endobronchial implant therapy, after endobronchial implant therapy, before administration of a bronchodilator, after administration of a bronchodilator, before exercise, or during exercise.

16. The method of any one of claims 1-15, wherein the predicted response comprises a prediction of one or more of the following: forced expiratory volume in 1 second, forced vital capacity, vital capacity, inspiratory capacity, inspiratory capacity/total lung capacity ratio, functional residual capacity, total lung capacity, diffusion capacity for carbon monoxide, residual volume, residual volume/total lung capacity ratio, segmental volume, mMRC score, SGRQ score or a subset thereof, CAT score or a subset thereof, 6-minute walk test results, cycle ergometry results, cardiopulmonary exercise testing (CPET) results, patient health metrics, patient exercise metrics, patient visit metrics, number of required implant removals, time to reintervention, durability of treatment, quality of life score, body mass index, comorbidities, drug regimen, length of hospitalization, healthcare utilization, or cost.

17. The method of any one of claims 1-16, wherein the treatment comprises an airway treatment for COPD.

18. The method of claim 17, wherein the airway treatment comprises a pharmacological treatment.

19. The method of claim 17 or 18, wherein the airway treatment comprises an interventional treatment.

20. The method of claim 19, wherein the interventional treatment comprises one or more of the following: vapor therapy, administration of a sealant, transbronchial fenestration, placement of an endobronchial coil, placement of an endobronchial valve, or placement of a minimal endobronchial reinforcement implant.

21. The method of claim 20, wherein the interventional treatment comprises the placement of the minimal endobronchial reinforcement implant.

22. The method of any one of claims 1-21, further comprising generating a plan for the treatment, if the patient is a candidate for the treatment with the pulmonary disease.

23. The method of claim 22, wherein the plan is generated by inputting one or more of the predicted response or the set of lung metrics into a third machine learning algorithm.

24. The method of claim 22 or 23, wherein the treatment comprises placement of at least one minimal endobronchial reinforcement implant, and the plan comprises one or more of the following: implant placement location, number of implants, implant size, implant type, pathway to a target location, or localized treatment solutions.

25. The method of claim 24, wherein the implant placement location is based at least in part on one of more of the following: location of dynamic airway collapse as determined from expiratory CT data, severity of disease in a peripheral region of the lung, location of a pleural wall of the patient, or location of lobar, segmental, and/or sub-segmental airways.

26. The method of any one of claims 1-25, further comprising generating a report comprising a summary of one or more of the following: at least a portion of the set of lung metrics, the predicted response, the evaluation of whether the patient is a candidate for the treatment, or the generated plan for the treatment.

27. The method of any one of claims 1-26, further comprising updating one or more of the first machine learning algorithm or the second machine learning algorithm based on historical or repository patient data.

28. The method of claim 27, wherein the historical or repository patient data comprises data of patients having GOLD III COPD, data of patients having GOLD IV COPD, or a combination thereof.

29. The method of claim 27 or 28, wherein the historical or repository patient data comprises data of patients treated with one or more of the following: a minimal endobronchial reinforcement implant, an endobronchial valve, an endobronchial coil, or vapor therapy.

30. The method of any one of claims 27-29, wherein the historical or repository patient data comprises data of the patient from an earlier time point.

31. A system comprising:
a processor; and
a memory operably coupled to the processor and storing instructions that, when executed by the processor, cause the computing system to perform operations comprising the method of any one of claims 1-30.

32. A non-transitory computer-readable storage medium comprising instructions that, when executed by one or more processors of a computing system, cause the computing system to perform operations comprising the method of any one of claims 1-30.

33. A method for evaluating a treatment outcome of a patient, the method comprising:
receiving patient data including computed tomography (CT) data of a lung of the patient after placement of an endobronchial implant in the lung;
generating a set of status metrics by inputting the patient data into a first machine learning algorithm, wherein the set of status metrics includes:
a set of lung metrics representing a state of the lung of the patient after the placement of the endobronchial implant, and
a set of implant metrics representing a state of the endobronchial implant after placement in the lung;
determining a response of the patient to the endobronchial implant by inputting the set of status metrics into a second machine learning algorithm; and
predicting an outcome of the patient after the placement of the endobronchial implant by inputting one or more of the set of status metrics or the determined response into a third machine learning algorithm.

34. The method of claim 33, wherein the patient data comprises one or more of the following: questionnaire information, medical record information, magnetic resonance imaging (MRI) data, single-photon emission computed tomography (SPECT) data, bronchoscopy data, ventilation-perfusion data, pulmonary function test data, chest radiography data, fluoroscopy data, photographs, or sensor data.

35. The method of claim 33 or 34, wherein the CT data comprises expiratory CT data.

36. The method of any one of claims 33-35, wherein the CT data comprises inspiratory CT data.

37. The method of any one of claims 33-36, wherein the patient data comprises data obtained at a plurality of different time points.

38. The method of any one of claims 33-37, wherein the set of lung metrics characterizes one or more lung parameters, the one or more lung parameters characterizing any of the following: forced expiratory volume in 1 second, forced vital capacity, vital capacity, inspiratory capacity, inspiratory capacity/total lung capacity ratio, functional residual capacity, total lung capacity, diffusion capacity for carbon monoxide, residual volume, residual volume/total lung capacity ratio, closing volume, lobar and/or segmental tissue destruction, lobar and/or segmental air trapping, lobar and/or segmental fissure status, extent of lobar and/or segmental fissure completion, lobar and/or segmental ventilation, lung function, homogeneity/heterogeneity of lobar and/or segmental emphysema, emphysema type, location of diseased portions of the lung, lobar volume, segmental volume, segment locations, diaphragm shape, tissue density, opacity, proximity of diseased portions to anatomical structures, proximity of disease portions to other medical devices, lumen inner diameter of bronchial sections, air flow mapping, collapsed airways, airway pressure, airway compliance, pleural pressure, airway diameter to wall thickness, airway wall deformation, vascular perfusion, parenchyma density, bullae, information that localizes disease in the lung, obstruction score, mucus score, degree of epithelialization, granulation tissue, implant-induced airway deformation, or airway tissue invagination into a lumen of the implant.

39. The method of any one of claims 33-38, wherein the set of lung metrics comprises a disease score characterizing severity of pulmonary disease in the patient.

40. The method of claim 38 or 39, wherein the set of lung metrics characterizes a change in at least one of the one or more lung parameters over a plurality of time points.

41. The method of claim 40, wherein the plurality of time points comprise two or more of the following: before endobronchial implant therapy, after endobronchial implant therapy, before administration of a bronchodilator, after administration of a bronchodilator, before exercise, or during exercise.

42. The method of any one of claims 33-41, wherein the endobronchial implant comprises a minimal endobronchial reinforcement implant.

43. The method of any one of claims 33-42, wherein the set of implant metrics characterizes one or more of the following: implant location, distance between a distal end of the implant and pleura, implant length, implant diameter at any one or more locations along a length of the implant, implant cross-sectional profile at any one or more locations along a length of the implant, implant integrity, pitch of loops of an implant, angle of an implant loop profile relative to a longitudinal axis of the implant, implant position relative to one or more additional implants, movement of the implant between inspiration and expiration, occlusion of the implant, or implant dislodgment.

44. The method of any one of claims 33-43, further comprising generating and displaying a virtual bronchoscopy depicting a model incorporating one or more of at least a portion of the lung metrics or at least a portion of the implant metrics.

45. The method of any one of claims 33-44, wherein the determined response comprises a determination of one or more of the following: forced expiratory volume in 1 second, forced vital capacity, vital capacity, inspiratory capacity, inspiratory capacity/total lung capacity ratio, functional residual capacity, total lung capacity, diffusion capacity for carbon monoxide, residual volume, residual volume/total lung capacity ratio, segmental volume, mMRC score, SGRQ score or a subset thereof, CAT score or a subset thereof, 6-minute walk test results, cycle ergometry results, cardiopulmonary exercise testing (CPET) results, patient health metrics, patient exercise metrics, patient visit metrics, number of required implant removals, time to reintervention, durability of treatment, quality of life score, body mass index, comorbidities, drug regimen, length of hospitalization, healthcare utilization, or cost.

46. The method of any one of claims 33-45, wherein the predicted outcome comprises a prediction of a post-procedure issue after the placement of the endobronchial implant.

47. The method of claim 46, wherein the post-procedure issue comprises one or more of the following: copious mucus, excessive granulation tissue, excessive fibrosis, implant collapse, implant failure, implant migration, implant expectoration, inadequate lung function, pneumothorax, infection, pneumonia, or hospitalization.

48. The method of claim 46 or 47, further comprising determining an intervention to address the post-procedure issue.

49. The method of claim 48, wherein the determined intervention comprises one or more of the following: cleanup bronchoscopy, retrieval or removal of the endobronchial implant, repositioning of the endobronchial implant, replacement of the endobronchial implant, dilation of the endobronchial implant, placement of an additional endobronchial implant, or consultation with a healthcare professional.

50. The method of any one of claims 33-49, further comprising generating a report comprising a summary of one or more of the following: at least a portion of the lung metrics, at least a portion of the implant metrics, the determined response of the patient to the endobronchial implant, the predicted outcome of the patient after the placement of the endobronchial implant, or the determined intervention to address a post-procedure issue.

51. The method of any one of claims 33-50, further comprising updating one or more of the first machine learning algorithm, the second machine learning algorithm, or the third machine learning algorithm based on historical or repository patient data.

52. The method of claim 51, wherein the historical or repository patient data comprises data of patients having GOLD III COPD, data of patients having GOLD IV COPD, or a combination thereof.

53. The method of claim 51 or 52, wherein the historical or repository patient data comprises data of patients treated with one or more of the following: a minimal endobronchial reinforcement implant, an endobronchial valve, an endobronchial coil, or vapor therapy.

54. The method of any one of claims 51-53, wherein the historical or repository patient data comprises data of the patient from an earlier time point.

55. The method of any one of claims 33-54, further comprising comparing the set of lung metrics to a set of second lung metrics determined from one or more of the following:
image data of the lung before the placement of the endobronchial implant, image data of the lung at an earlier time point after the placement of the endobronchial implant, image data of the lung after placement of another endobronchial implant at a different location than a location of the endobronchial implant, or image data from other patients having COPD.

56. A system comprising:
a processor; and
a memory operably coupled to the processor and storing instructions that, when executed by the processor, cause the computing system to perform operations comprising the method of any one of claims 33-55.

57. A non-transitory computer-readable storage medium comprising instructions that, when executed by one or more processors of a computing system, cause the computing system to perform operations comprising the method of any one of claims 33-55.

58. A method for evaluating a patient having or suspected of having a pulmonary disease, the method comprising:
receiving patient data including computed tomography (CT) data of a lung of the patient; and
generating a pulmonary disease score for a region of interest of the lung by inputting the patient data into a machine learning algorithm, wherein the pulmonary disease score characterizes a severity of pulmonary disease in the region of interest of the lung of the patient, wherein the region of interest is a segmental region or a sub-segmental region of the lung.

59. The method of claim 58, wherein the machine learning algorithm evaluates voxel density in the CT data associated with the region of interest of the lung of the patient.

60. The method of claim 58 or 59, wherein the pulmonary disease score is based on multiple local disease scores each corresponding to a respective lobar, segmental, or sub-segmental region of the lung of the patient.

61. The method of claim 60, wherein the pulmonary disease score is an average of the multiple local disease scores.

62. The method of claim 58 or 59, wherein the pulmonary disease score is a first pulmonary disease score, wherein the method further comprises generating a plurality of pulmonary disease scores comprising the first pulmonary disease score, wherein each of the plurality of pulmonary disease scores corresponds to a respective lobar, segmental, or sub-segmental region of the lung of the patient.

63. The method of any one of claims 58-62, wherein the pulmonary disease score represents an extent of at least one of air trapping or hyperinflation in the lung of the patient.

64. The method of any one of claims 58-63, wherein the CT data comprises expiratory CT data.

65. The method of any one of claims 58-64, wherein the CT data comprises inspiratory CT data.

66. The method of any one of claims 58-65, wherein the CT data is generated prior to a treatment administered to the patient to treat the pulmonary disease.

67. The method of any one of claims 58-65, wherein the CT data is generated following a treatment administered to the patient to treat the pulmonary disease.

68. The method of claim 66 or 67, wherein the treatment comprises placement of an endobronchial implant.

69. A system comprising:
a processor; and
a memory operably coupled to the processor and storing instructions that, when executed by the processor, cause the computing system to perform operations comprising the method of any one of claims 58-68.

70. A computed tomography (CT) scanner comprising:
a processor; and
a memory operably coupled to the processor and storing instructions that, when executed by the processor, cause the CT scanner to perform operations comprising the method of any one of claims 58-68.

71. A non-transitory computer-readable storage medium comprising instructions that, when executed by one or more processors of a computing system, cause the computing system to perform operations comprising the method of any one of claims 58-68.

72. A method for normalizing quantitative computed tomography (CT) results for a patient, the method comprising:
receiving first CT data for the patient, wherein the first CT data is generated under predetermined imaging conditions; and
transforming the first CT data to second CT data by applying to the first CT data at least one correction factor associated with the predetermined imaging conditions.

73. The method of claim 72, wherein the at least one correction factor maps voxel density in the first CT data to a normalized voxel density.

74. The method of claim 72 or 73, wherein the at least one correction factor compensates for voxel density in the first CT data affected by one or more of the following: tube current, tube potential, pitch,

75. The method of any one of claims 72-74, wherein the at least one correction factor compensates for voxel density in the first CT data affected by at least one of slice thickness or slice interval.

76. The method of any one of claims 72-75, wherein the at least one correction factor compensates for voxel density in the first CT data affected by a reconstruction algorithm for determining sharpness or smoothness of image in an axial plane.

77. The method of any one of claims 72-76, wherein the first CT data is obtained from a CT scan provider having a provider-specific machine learning algorithm for reconstructing a CT image from CT data, wherein the at least one correction factor compensates for voxel density in the first CT data affected by the provider-specific machine learning algorithm.

78. The method of any one of claims 72-77, wherein the at least one correction factor compensates for voxel density in the first CT data affected by administration of a contrast agent in the patient before the first CT data is generated.

79. The method of any one of claims 72-78, wherein the second CT data is normalized with respect to CT scan parameters.

80. The method of any one of claims 72-79, wherein the first CT data is obtained during a pre-procedure phase prior to placement of an endobronchial implant in the patient.

81. The method of any one of claims 72-80, further comprising generating a set of lung metrics associated with the patient based on the second CT data.

82. The method of any one of claims 72-79, wherein the first CT data is obtained during a peri-procedure phase during placement of an endobronchial implant in the patient.

83. The method of any one of claims 72-79, wherein the first CT data is obtained during a post-procedure phase following placement of an endobronchial implant in the patient.

84. The method of claim 82 or 83, further comprising generating at least one of a set of lung metrics or a set of implant metrics associated with the patient based on the second CT data.

85. A system comprising:
a processor; and
a memory operably coupled to the processor and storing instructions that, when executed by the processor, cause the computing system to perform operations comprising the method of any one of claims 72-84.

86. A computed tomography (CT) scanner comprising:
a processor; and
a memory operably coupled to the processor and storing instructions that, when executed by the processor, cause the CT scanner to perform operations comprising the method of any one of claims 72-84.

87. A non-transitory computer-readable storage medium comprising instructions that, when executed by one or more processors of a computing system, cause the computing system to perform operations comprising the method of any one of claims 72-84.

88. A method for planning a treatment for a patient having a pulmonary disease, the method comprising:
receiving patient data including computed tomography (CT) data of a lung of the patient;
generating a set of lung metrics by inputting the patient data into a first machine learning algorithm, wherein the set of lung metrics represents a state of the lung of the patient;
identifying a potential target region in the lung for a treatment for the pulmonary disease, based at least in part on the generated lung metrics.

89. The method of claim 88, wherein the patient data comprises one or more of the following: questionnaire information, medical record information, magnetic resonance imaging (MRI) data, single-photon emission computed tomography (SPECT) data, bronchoscopy data, ventilation-perfusion data, pulmonary function test data, chest radiography data, fluoroscopy data, photographs, or sensor data.

90. The method of claim 88 or 89, wherein the CT data comprises expiratory CT data.

91. The method of any one of claims 88-90, wherein the CT data comprises inspiratory CT data.

92. The method of any one of claims 88-91, wherein the patient data comprises data obtained at a plurality of different time points.

93. The method of any one of claims 88-92, wherein the set of lung metrics correlates to whether the patient has at least one of chronic obstructive pulmonary disease (COPD), severe emphysema, or severe emphysema with hyperinflation.

94. The method of any one of claims 88-93, wherein the set of lung metrics characterizes one or more lung parameters, the one or more lung parameters comprising one or more of the following: forced expiratory volume in 1 second, forced vital capacity, vital capacity, inspiratory capacity, inspiratory capacity/total lung capacity ratio, functional residual capacity, total lung capacity, diffusion capacity for carbon monoxide, residual volume, residual volume/total lung capacity ratio, closing volume, lobar and/or segmental tissue destruction, lobar and/or segmental air trapping, lobar and/or segmental fissure status, extent of lobar and/or segmental fissure completion, lobar and/or segmental ventilation, lung function, homogeneity/heterogeneity of lobar and/or segmental emphysema, emphysema type, locations of diseased portions of the lung, lobar volume, segmental volume, segmental locations, diaphragm shape, tissue density, opacity, proximity of diseased portions to anatomical structures, proximity of diseased portions to other medical devices, lumen inner diameter of bronchial sections, air flow mapping, collapsed airways, airway pressure, airway compliance, pleural pressure, airway diameter to wall thickness, airway wall deformation, vascular perfusion, parenchyma density, bullae, information that localizes disease in the lung, obstruction score, mucus score, or degree of epithelialization.

95. The method of any one of claims 88-94, wherein the set of lung metrics comprises at least one disease score characterizing severity of pulmonary disease in the patient.

96. The method of claim 95, wherein the at least one disease score represents a predictor of patient response to the treatment of the pulmonary disease.

97. The method of claim 95 or 96, wherein the set of lung metrics comprises multiple disease scores each corresponding to a respective lobar, segmental, or sub-segmental region of the lung of the patient.

98. The method of claim 95 or 96, wherein the set of lung metrics comprises a single disease score based on multiple local disease scores each corresponding to a respective lobar, segmental, or sub-segmental region of the lung of the patient.

99. The method of claim 98, wherein the single disease score is an average of the multiple local disease scores.

100. The method of any one of claims 95-99, wherein the at least one disease score represents an extent of at least one of air trapping or hyperinflation in the lung of the patient.

101. The method of any one of claims 94-100, wherein the set of lung metrics characterizes a change in at least one of the one or more lung parameters over a plurality of time points.

102. The method of claim 101, wherein the plurality of time points comprise two or more of the following: before endobronchial implant therapy, after endobronchial implant therapy, before administration of a bronchodilator, after administration of a bronchodilator, before exercise, or during exercise.

103. The method of any one of claims 88-102, wherein the treatment comprises an airway treatment for COPD.

104. The method of claim 103, wherein the airway treatment comprises a pharmacological treatment.

105. The method of claim 103 or 104, wherein the airway treatment comprises an interventional treatment.

106. The method of claim 105, wherein the interventional treatment comprises one or more of the following: vapor therapy, administration of a sealant, transbronchial fenestration, placement of an endobronchial coil, placement of an endobronchial valve, or placement of a minimal endobronchial reinforcement implant.

107. The method of claim 106, wherein the interventional treatment comprises the placement of the minimal endobronchial reinforcement implant.

108. The method of any one of claims 88-107, further comprising generating a plan for the treatment.

109. The method of claim 108, wherein the plan is generated by inputting the set of lung metrics into a second machine learning algorithm.

110. The method of claim 108 or 109, wherein the treatment comprises placement of at least one minimal endobronchial reinforcement implant, and the plan comprises one or more of the following: implant placement location, number of implants, implant size, implant type, pathway to a target location, or localized treatment solutions.

111. The method of claim 110, wherein the implant placement location is based at least in part on one of more of the following: location of dynamic airway collapse as determined from expiratory CT data, severity of disease in a peripheral region of the lung, location of a pleural wall of the patient, or location of lobar, segmental, and/or sub-segmental airways.

112. A system comprising:
a processor; and
a memory operably coupled to the processor and storing instructions that, when executed by the processor, cause the computing system to perform operations comprising the method of any one of claims 88-111.

113. A computed tomography (CT) scanner comprising:
a processor; and
a memory operably coupled to the processor and storing instructions that, when executed by the processor, cause the CT scanner to perform operations comprising the method of any one of claims 88-111.

114. A non-transitory computer-readable storage medium comprising instructions that, when executed by one or more processors of a computing system, cause the computing system to perform operations comprising the method of any one of claims 88-111.
